SEQUENCE LISTING (22 sequences)

SEQ ID NO: 1 - DNA, 37 bp, synthetic construct
  source 1..37: mol_type = other DNA
  misc_feature 37: note = adenine attached to Cy3B, which is joined to another nucleic acid
cggatttattcatagcttgtgctatgtggcatcgata

SEQ ID NO: 2 - DNA, 37 bp, synthetic construct
  source 1..37: mol_type = other DNA
  misc_feature 37: note = thymine attached to ATRho6G, which is joined to another nucleic acid
cgcttattatcgatgccacatagcacaagctatgaat

SEQ ID NO: 3 - DNA, 38 bp, synthetic construct
  source 1..38: mol_type = other DNA
  misc_feature 38: note = thymine attached to Cy3, which is joined to another nucleic acid
ggctatttatgtatgagttcatgtgatgcgagctatat

SEQ ID NO: 4 - DNA, 39 bp, synthetic construct
  source 1..39: mol_type = other DNA
  misc_feature 39: note = adenine attached to Cy3B, which is joined to another nucleic acid
ccgttgccttatagctcgcatcacatgaactcatacata

SEQ ID NO: 5 - DNA, 24 bp, synthetic construct
  source 1..24: mol_type = other DNA
  misc_feature 1: note = thymine attached to Cy3, which is joined to another nucleic acid
tgccgttgcctcgacagatcccga

SEQ ID NO: 6 - DNA, 13 bp, synthetic construct
  source 1..13: mol_type = other DNA
  misc_feature 1: note = thymine attached to Cy3, which is joined to another nucleic acid
  misc_feature 13: note = thymine attached to Cy3, which is joined to another nucleic acid
tgatctgtcgagt

SEQ ID NO: 7 - DNA, 19 bp, synthetic construct
  source 1..19: mol_type = other DNA
  misc_feature 1: note = thymine attached to Cy3, which is joined to another nucleic acid
tgcaacggccgtgccgcct

SEQ ID NO: 8 - DNA, 26 bp, synthetic construct
  source 1..26: mol_type = other DNA
  misc_feature 1: note = adenine attached to C530NS, which is joined to another nucleic acid
attcggttatattgcctattattgcg

SEQ ID NO: 9 - DNA, 26 bp, synthetic construct
  source 1..26: mol_type = other DNA
  misc_feature 1: note = thymine attached to C530NS, which is joined to another nucleic acid
taaccgaattaatccgtattaatggc

SEQ ID NO: 10 - DNA, 26 bp, synthetic construct
  source 1..26: mol_type = other DNA
  misc_feature 1: note = thymine attached to C530NS, which is joined to another nucleic acid
  modified_base 5: mod_base = OTHER; note = isocytosine
  modified_base 25: mod_base = OTHER; note = isoguanine
taagncaaataatgcgtaataaagnc

SEQ ID NO: 11 - DNA, 26 bp, synthetic construct
  source 1..26: mol_type = other DNA
  misc_feature 1: note = thymine attached to C530NS, which is joined to another nucleic acid
  modified_base 5: mod_base = OTHER; note = isoguanine
  modified_base 15: mod_base = OTHER; note = isocytosine
  modified_base 25: mod_base = OTHER; note = isoguanine
  modified_base 26: mod_base = OTHER; note = isocytosine
tttgncttatatacncttatatacnn

SEQ ID NO: 12 - DNA, 19 bp, synthetic construct
  source 1..19: mol_type = other DNA
  misc_feature 1: note = thymine attached to Cy3 phosphoramidite, which is joined to another nucleic acid
taccgatatgaatcttgcg

SEQ ID NO: 13 - DNA, 17 bp, synthetic construct
  source 1..17: mol_type = other DNA
  misc_feature 1: note = thymine attached to Cy3 phosphoramidite, which is joined to another nucleic acid
ttatcggttgggttcgg

SEQ ID NO: 14 - DNA, 22 bp, synthetic construct
  source 1..22: mol_type = other DNA
  misc_feature 1: note = thymine attached to internal amine phosphoramidite, which is joined to another nucleic acid
ttacatccgactacagttacct

SEQ ID NO: 15 - DNA, 10 bp, synthetic construct
  source 1..10: mol_type = other DNA
  misc_feature 10: note = thymine attached to Cy3 phosphoramidite, which is joined to another nucleic acid
agcgaggtat

SEQ ID NO: 16 - DNA, 19 bp, synthetic construct
  source 1..19: mol_type = other DNA
  misc_feature 1: note = thymine attached to Cy3 phosphoramidite, which is joined to another nucleic acid
tagtcggatgtaattatcg

SEQ ID NO: 17 - protein, 9 aa, synthetic construct
FAAAYPDDD

SEQ ID NO: 18 - protein, 11 aa, synthetic construct
RLIFAYPDDDK

SEQ ID NO: 19 - protein, 7 aa, synthetic construct
RLIFAGK

SEQ ID NO: 20 - protein, 9 aa, synthetic construct
DQLRLAGGK

SEQ ID NO: 21 - protein, 5 aa, synthetic construct
RLIFA

SEQ ID NO: 22 - protein, 4 aa, synthetic construct
LIFA

(19) United States
(12) Patent Application Publication
(10) Pub. No.: US 2024/0151729 A1
(43) Pub. Date: May 9, 2024

(54) LUMINESCENTLY LABELED OLIGONUCLEOTIDE STRUCTURES AND ASSOCIATED SYSTEMS AND METHODS

(71) Applicant: Quantum-Si Incorporated, Branford, CT (US)

(72) Inventors: Haidong Huang, Madison, CT (US); Saketh Gudipati, East Haven, CT (US); Roger R. Nani, Madison, CT (US)

(73) Assignee: Quantum-Si Incorporated, Branford, CT (US)

(21) Appl. No.: 18/491,693

(22) Filed: Oct. 20, 2023

Related U.S. Application Data
(60) Provisional application No. 63/418,308, filed on Oct. 21, 2022.

(51) Int. Cl. G01N 33/58; G01N 33/68

(52) U.S. Cl. CPC G01N 33/582; G01N 33/6803

(57) ABSTRACT

Provided herein are luminescently labeled oligonucleotide structures, which may be used in systems and methods for polypeptide sequencing and/or nucleic acid sequencing.

### CROSS-REFERENCE TO RELATED APPLICATIONS

This application claims priority under 35 U.S.C. § 119(e) to U.S. Provisional Patent Application No. 63/418,308, filed Oct. 21, 2022, which is hereby incorporated by reference in its entirety.

### REFERENCE TO AN ELECTRONIC SEQUENCE LISTING

The contents of the electronic sequence listing (R070870141US01-SEQ-JIB.xml; Size: 30,925 bytes; and Date of Creation: Oct. 20, 2023) is herein incorporated by reference in its entirety.

### FIELD

Luminescently labeled oligonucleotide structures and associated systems and methods are generally described.

### BACKGROUND

Luminescent labels are often used in systems and methods for detecting and/or characterizing biological analytes. Some of these systems and methods involve monitoring a biological reaction in real time using a plurality of types of luminescently labeled reaction components. In order to identify specific types of luminescently labeled reaction components, it is important that each type of reaction component be labeled with a luminescent label having readily differentiable luminescent properties. However, the sensitivity of complex biological processes requires careful consideration when designing luminescent labels for use in these systems and methods.

### SUMMARY

Luminescently labeled oligonucleotide structures and associated systems and methods are generally described. The subject matter disclosed herein involves, in some cases, interrelated products, alternative solutions to a particular problem, and/or a plurality of different uses of one or more systems and/or articles.

In some aspects, a luminescently labeled oligonucleotide structure is provided. In some embodiments, the structure comprises a first single-stranded oligonucleotide comprising one or more first luminescent labels. In some embodiments, the structure comprises a first complementary single-stranded oligonucleotide hybridized to the first single-stranded oligonucleotide. In certain embodiments, the first complementary single-stranded oligonucleotide comprises one or more second luminescent labels. In certain embodiments, a closest distance between any first luminescent label and any second luminescent label is at least 10 nm.

In some aspects, a luminescently labeled oligonucleotide structure is provided. In some embodiments, the structure comprises a first single-stranded oligonucleotide comprising two or more first luminescent labels. In some embodiments, the structure comprises a first complementary single-stranded oligonucleotide hybridized to the first single-stranded oligonucleotide. In certain embodiments, the first complementary single-stranded oligonucleotide comprises two or more first luminescent labels. In certain embodiments, the first luminescent label comprises a cyanine dye.

In some aspects, a luminescently labeled oligonucleotide structure is provided. In some embodiments, the structure comprises a first single-stranded oligonucleotide bound to a first binding molecule (e.g., a multivalent protein, such as an avidin protein). In some embodiments, the structure comprises a first complementary single-stranded oligonucleotide hybridized to the first single-stranded oligonucleotide. In some embodiments, the structure comprises a second single-stranded oligonucleotide bound to the first binding molecule. In some embodiments, the structure comprises a second complementary single-stranded oligonucleotide hybridized to the second single-stranded oligonucleotide. In certain embodiments, the first single-stranded oligonucleotide and/or the first complementary single-stranded oligonucleotide are conjugated to one or more first luminescent labels. In certain embodiments, the second single-stranded oligonucleotide and/or the second complementary single-stranded oligonucleotide are conjugated to one or more second luminescent labels.

In some aspects, a system is provided. In some embodiments, the system comprises an integrated device comprising a plurality of sample wells. In certain embodiments, one or more sample wells are adapted to have a polypeptide immobilized to a surface thereof. In some embodiments, the system comprises one or more first amino acid recognition molecules bound to a first luminescent label comprising a first luminescently labeled oligonucleotide structure. In certain embodiments, the first luminescently labeled oligonucleotide structure comprises a first single-stranded oligonucleotide comprising one or more first fluorophores. In certain embodiments, the first luminescently labeled oligonucleotide structure comprises a first complementary single-stranded oligonucleotide hybridized to the first single-stranded oligonucleotide. In some cases, the first complementary single-stranded oligonucleotide comprises one or more second fluorophores. In some cases, a closest distance between any first luminescent label and any second luminescent label is at least 10 nm.

In some aspects, a system is provided. In some embodiments, the system comprises an integrated device comprising a plurality of sample wells. In certain embodiments, one or more sample wells are adapted to have a polypeptide immobilized to a surface thereof. In some embodiments, the system comprises one or more first amino acid recognition molecules bound to a first luminescent label comprising a luminescently labeled oligonucleotide structure. In certain embodiments, the luminescently labeled oligonucleotide structure comprises a first single-stranded oligonucleotide comprising two or more first luminescent labels. In certain embodiments, the luminescently labeled oligonucleotide structure comprises a first complementary single-stranded oligonucleotide hybridized to the first single-stranded oligonucleotide. In some cases, the first complementary single-stranded oligonucleotide comprises two or more first fluorophores. In some cases, the first luminescent label comprises a cyanine dye.

In some aspects, a system is provided. In some embodiments, the system comprises an integrated device comprising a plurality of sample wells. In certain embodiments, one or more sample wells are adapted to have a polypeptide immobilized to a surface thereof. In some embodiments, the system comprises one or more first amino acid recognition molecules bound to a first luminescent label comprising a luminescently labeled oligonucleotide structure. In certain embodiments, the luminescently labeled oligonucleotide structure comprises a first single-stranded oligonucleotide bound to a first binding molecule (e.g., a multivalent protein, such as an avidin protein). In certain embodiments, the luminescently labeled oligonucleotide structure comprises a first complementary single-stranded oligonucleotide hybridized to the first single-stranded oligonucleotide. In certain embodiments, the luminescently labeled oligonucleotide structure comprises a second single-stranded oligonucleotide bound to the first binding molecule. In certain embodiments, the luminescently labeled oligonucleotide structure comprises a second complementary single-stranded oligonucleotide hybridized to the second single-stranded oligonucleotide. In some cases, the first single-stranded oligonucleotide and/or the first complementary single-stranded oligonucleotide are conjugated to one or more first fluorophores. In some cases, the second single-stranded oligonucleotide and/or the second complementary single-stranded oligonucleotide are conjugated to one or more second fluorophores.

In some aspects, a method for determining chemical characteristics of a polypeptide is provided. In some embodiments, the method comprises contacting a polypeptide with one or more first amino acid recognition molecules bound to a first luminescent label comprising a luminescently labeled oligonucleotide structure. In certain embodiments, the luminescently labeled oligonucleotide structure comprises a first single-stranded oligonucleotide comprising one or more first fluorophores. In certain embodiments, the luminescently labeled oligonucleotide structure comprises a first complementary single-stranded oligonucleotide hybridized to the first single-stranded oligonucleotide. In some cases, the first complementary single-stranded oligonucleotide comprises one or more second fluorophores. In some cases, a closest distance between any first fluorophore and any second fluorophore is at least 10 nm. In some embodiments, the method comprises detecting a first series of signal pulses indicative of a first series of binding events between the one or more amino acid recognition molecules and the polypeptide. In some embodiments, the method comprises determining at least one chemical characteristic for an amino acid of the polypeptide based on at least one characteristic of the first series of signal pulses.

In some aspects, a method for determining chemical characteristics of a polypeptide is provided. In some embodiments, the method comprises contacting a polypeptide with one or more first amino acid recognition molecules bound to a first luminescent label comprising a luminescently labeled oligonucleotide structure. In certain embodiments, the luminescently labeled oligonucleotide structure comprises a first single-stranded oligonucleotide comprising two or more first fluorophores. In certain embodiments, the luminescently labeled oligonucleotide structure comprises a first complementary single-stranded oligonucleotide hybridized to the first single-stranded oligonucleotide. In some cases, the first complementary single-stranded oligonucleotide comprises two or more first fluorophores. In some cases, the first luminescent label comprises a cyanine dye. In some embodiments, the method comprises detecting a first series of signal pulses indicative of a first series of binding events between the one or more amino acid recognition molecules and the polypeptide. In some embodiments, the method comprises determining at least one chemical characteristic for an amino acid of the polypeptide based on at least one characteristic of the first series of signal pulses.

In some aspects, a method for determining chemical characteristics of a polypeptide is provided. In some embodiments, the method comprises contacting a polypeptide with one or more first amino acid recognition molecules bound to a first luminescent label comprising a luminescently labeled oligonucleotide structure. In certain embodiments, the luminescently labeled oligonucleotide comprises a first single-stranded oligonucleotide bound to a first binding molecule (e.g., a multivalent protein, such as an avidin protein). In certain embodiments, the luminescently labeled oligonucleotide comprises a first complementary single-stranded oligonucleotide hybridized to the first single-stranded oligonucleotide. In certain embodiments, the luminescently labeled oligonucleotide comprises a second single-stranded oligonucleotide bound to the first binding molecule. In certain embodiments, the luminescently labeled oligonucleotide comprises a second complementary single-stranded oligonucleotide hybridized to the second single-stranded oligonucleotide. In some cases, the first single-stranded oligonucleotide and/or the first complementary single-stranded oligonucleotide are conjugated to one or more first fluorophores. In some cases, the second single-stranded oligonucleotide and/or the second complementary single-stranded oligonucleotide are conjugated to one or more second fluorophores. In some embodiments, the method comprises detecting a first series of signal pulses indicative of a first series of binding events between the one or more amino acid recognition molecules and the polypeptide. In some embodiments, the method comprises determining at least one chemical characteristic for an amino acid of the polypeptide based on at least one characteristic of the first series of signal pulses.

In some aspects, a system is provided. In some embodiments, the system comprises a first luminescent label having a first ordered pair of characteristics comprising a first value of a first characteristic and a first value of a second characteristic. In some embodiments, the system comprises a second luminescent label having a second ordered pair of characteristics comprising a second value of the first characteristic and a second value of the second characteristic. In some embodiments, the system comprise a third luminescent label having a third ordered pair of characteristics comprising a third value of the first characteristic and a third value of the second characteristic. In certain embodiments, the first ordered pair, the second ordered pair, and the third ordered pair differ from one another in at least one of the respective values of the first and/or second characteristics.

In some aspects, a method is provided. In some embodiments, the method comprises providing a first luminescent label having a first ordered pair of characteristics comprising a first value of a first characteristic and a first value of a second characteristic. In some embodiments, the method comprises providing a second luminescent label having a second ordered pair of characteristics comprising a second value of the first characteristic and a second value of the second characteristic. In some embodiments, the method comprise providing a third luminescent label comprising a luminescently labeled oligonucleotide structure comprising a first single-stranded oligonucleotide comprising one or more first fluorophores and a first complementary single-stranded oligonucleotide comprising one or more second fluorophores. In certain embodiments, the third luminescent label has a third ordered pair of characteristics comprising a third value of the first characteristic and a third value of the second characteristic. In some embodiments, the method comprises modifying the numbers and/or identities of the one or more first fluorophores and/or the one or more second fluorophores such that the first ordered pair, the second ordered pair, and the third ordered pair differ from one another in at least one of the respective values of the first and/or second characteristics.

In some aspects, a system is provided. In some embodiments, the system comprises a first luminescent label having a first bin ratio value. In some embodiments, the system comprises a second luminescent label having a second bin ratio value. In some embodiments, the system comprises a third luminescent label having a third bin ratio value. In certain embodiments, a minimum difference between the first bin ratio value, the second bin ratio value, and the third bin ratio value of the first luminescence characteristic is at least 0.1.

The details of certain embodiments of the invention are set forth in the Detailed Description of Certain Embodiments, as described below. Other features, objects, and advantages of the invention will be apparent from the Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1A shows, according to some embodiments, a schematic illustration of an exemplary luminescently labeled oligonucleotide structure comprising a first single-stranded oligonucleotide comprising one copy of a first luminescent label and a first complementary single-stranded oligonucleotide comprising one copy of a second luminescent label.

FIG. 1B shows, according to some embodiments, a schematic illustration of the exemplary luminescently labeled oligonucleotide structure of FIG. 1A bound to a reaction component (e.g., an amino acid recognition molecule, a nucleotide) through a binding molecule.

FIG. 2A shows a schematic illustration of an exemplary luminescently labeled oligonucleotide structure comprising a first single-stranded oligonucleotide comprising two copies of a first luminescent label and a first complementary single-stranded oligonucleotide comprising one copy of a second luminescent label, according to some embodiments.

FIG. 2B shows a schematic illustration of the exemplary luminescently labeled oligonucleotide structure of FIG. 2A bound to a reaction component (e.g., an amino acid recognition molecule, a nucleotide) through a binding molecule, according to some embodiments.

FIG. 3A shows, according to some embodiments, a schematic illustration of an exemplary method of assembling a luminescently labeled oligonucleotide structure.

FIG. 3B shows, according to some embodiments, a schematic illustration of an exemplary method of assembling a luminescently labeled oligonucleotide structure by ligation.

FIG. 4 shows an example overview of real-time dynamic protein sequencing, according to some embodiments. Protein samples are digested into peptide fragments, immobilized in nanoscale reaction chambers, and incubated with a mixture of freely-diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs). From left to right, SEQ ID NOs: 21 and 22 are shown.

FIG. 5 shows, according to some embodiments, an example schematic of a pixel of an integrated device.

FIG. 6A shows a representative trace from a polypeptide sequencing reaction performed for a sample peptide (FAAAYPDDD (SEQ ID NO: 17)) using amino acid recognition molecules labeled with a first luminescent label comprising 4 copies of Cy®3B, a second luminescent label comprising 3 copies of ATRho6G, and a third luminescent label comprising a luminescently labeled oligonucleotide structure comprising a first single-stranded oligonucleotide comprising one copy of Cy®3B and having 100% sequence identity to Sequence A and a first complementary single-stranded oligonucleotide comprising one copy of ATRho6G and having 100% sequence identity to Sequence B (referred to as R1C1).

FIG. 6B shows a plot of intensity v. bin ratio for the polypeptide sequencing reaction of FIG. 6A.

FIG. 7A shows a representative trace from a polypeptide sequencing reaction performed for a sample peptide (FAAAYPDDD (SEQ ID NO: 17)) using amino acid recognition molecules labeled with a first luminescent label comprising 8 copies of Cy®3, a second luminescent label comprising 4 copies of Cy®3B, and a third luminescent label comprising a luminescently labeled oligonucleotide structure comprising a first single-stranded oligonucleotide comprising two copies of Cy®3 and having 100% sequence identity to Sequence C and a first complementary single-stranded oligonucleotide comprising one copy of Cy®3B and having 100% sequence identity to Sequence D (referred to as C2C).

FIG. 7B shows a plot of intensity v. bin ratio for the polypeptide sequencing reaction of FIG. 7A.

FIG. 8A shows a representative trace from a polypeptide sequencing reaction performed for a sample peptide (FAAAYPDDD (SEQ ID NO: 17)) using amino acid recognition molecules labeled with a first luminescent label comprising 4 copies of Cy®3B, a second luminescent label comprising C2C, and a third luminescent label comprising a luminescently labeled oligonucleotide structure comprising a first single-stranded oligonucleotide comprising two copies of Cy®3 and having 100% sequence identity to Sequence E and a first complementary single-stranded oligonucleotide comprising two copies of Cy®3 and having 100% sequence identity to Sequence F (referred to as SG4Cy®3).

FIG. 8B shows a plot of intensity v. bin ratio for the polypeptide sequencing reaction of FIG. 8A.

FIG. 9A shows a retention plot illustrating the first step of assembly of luminescently labeled oligonucleotides: conjugating a first luminescently labeled oligonucleotide strand (ODN1) bound to streptavidin (SV) to a second luminescently labeled oligonucleotide strand (ODN3).

FIG. 9B shows a retention plot illustrating the second step of assembly of luminescently labeled oligonucleotides: hybridizing to ODN3 in the product of Step 1 shown in FIG. 9A a third luminescently labeled oligonucleotide strand (ODN4) that is bound to a second streptavidin, and hybridizing a complementary oligonucleotide strand (ODN2) to the first luminescently labeled oligonucleotide strand (ODN1).

FIG. 9C shows a retention plot illustrating the final step of assembly of luminescently labeled oligonucleotides: conjugating the product of Step 2 shown in FIG. 9B to an amino acid recognizer molecule (PS610).

FIG. 10A shows a representative trace from a polypeptide sequencing reaction performed for a sample peptide (RLIFAYPDDDK (SEQ ID NO: 18)) using a first luminescent label comprising 4 copies of Cy®3, a second luminescent label comprising 3 copies of Cy®3B, and a third luminescent label comprising 3 copies of ATRho6G.

FIG. 10B shows a representative trace from a polypeptide sequencing reaction performed for a sample peptide (RLIFAGK (SEQ ID NO: 19)) using a first luminescent label comprising 4 copies of Cy®3, a second luminescent label comprising 3 copies of Cy®3B, and a third luminescent label comprising 3 copies of C530NS.

FIG. 10C shows a representative trace from a polypeptide sequencing reaction performed for a sample peptide (RLIFAYPDDDK (SEQ ID NO: 18)) using a first luminescent label comprising 4 copies of Cy®3, a second luminescent label comprising 4 copies of Cy®3B, a third luminescent label comprising 2 copies of ATRho6G, and the luminescently labeled oligonucleotide structures from Example 4 comprising 8 copies of C530NS.

FIG. 11A shows a representative trace from a polypeptide sequencing reaction performed for a sample peptide (FAAAYPDDD (SEQ ID NO: 17)) using amino acid recognition molecules labeled with a first luminescent label comprising 4 copies of Cy®3 (referred to as “TetraCy3”), a second luminescent label comprising 4 copies of Cy®3B (referred to as “TetraCy3B”), and a third luminescent label comprising 8 copies of Cy®3 (referred to as “OctaCy3”).

FIG. 11B shows a plot of intensity v. bin ratio for the polypeptide sequencing reaction of FIG. 8A.

FIG. 12A shows a schematic illustration of an exemplary method that was used to assemble a luminescently labeled oligonucleotide structure by ligation and streptavidin conjugation.

FIG. 12B shows results from size-exclusion chromatography (top) and urea PAGE gel analysis (bottom) showing purification of a ligation product prepared according to FIG. 12A.

FIG. 12C shows results from size-exclusion chromatography showing purification of a streptavidin-conjugated product prepared according to FIG. 12A.

FIG. 12D shows results from size-exclusion chromatography showing purification of a streptavidin-conjugated amino acid recognition molecule prepared according to FIG. 12A.

FIG. 13A shows a representative trace (top) demonstrating phenylalanine recognition for a sample peptide (FAAAYPDDD (SEQ ID NO: 17)), and a plot of intensity vs. bin ratio (bottom) showing distinct spatial separation of LC6IF, C2C (Example 2), and a recognition molecule having 4 copies of Cy®3B (“4-Cy3B”).

FIG. 13B shows a representative trace (top) demonstrating phenylalanine recognition for a sample peptide (FAAAYPDDD (SEQ ID NO: 17)), and a plot of intensity vs. bin ratio (bottom) showing distinct spatial separation of LC6IF, C2C, and SG4Cy3 (Example 3).

FIG. 13C shows a representative trace (top) demonstrating amino acid recognition during sample peptide (DQLRLAGGK (SEQ ID NO: 20)) degradation, and a plot of intensity vs. bin ratio (bottom) showing distinct spatial separation of LC6IF and SG4Cy3.

FIG. 13D shows a representative trace (top) demonstrating amino acid recognition during sample peptide (DQLRLAGGK (SEQ ID NO: 20)) degradation, and a plot of intensity vs. bin ratio (bottom) showing distinct spatial separation of LC6IF, R1C1 (Example 1), and 4-Cy3B.

FIG. 13E shows a plot of intensity vs. bin ratio for a dye set comprising C2C, 4-Cy3B, and a label having 8 copies of Cy®3 in a construct prepared by ligation (“L8Cy3”).

FIG. 13F shows a plot of intensity vs. bin ratio for a dye set comprising C2C, 4-Cy3B, and a label having 8 copies of Cy®3 in a construct prepared by double-streptavidin linkage (“8Cy3”).

FIG. 13G shows a plot of intensity vs. bin ratio (top) and a table of corresponding values (bottom) for L8Cy3, LC6C, and LC6IF.

FIG. 13H shows a representative trace (top) demonstrating phenylalanine recognition for a sample peptide (FAAAYPDDD (SEQ ID NO: 17)), and a plot of intensity vs. bin ratio (bottom) showing distinct spatial separation of seven distinctly labeled recognition molecules.

FIG. 13I shows a representative trace (top) demonstrating amino acid recognition during sample peptide (DQLRLAGGK (SEQ ID NO: 20)) degradation, and a plot of intensity vs. bin ratio (bottom) showing distinct spatial separation of seven distinctly labeled recognition molecules.

### DETAILED DESCRIPTION

Luminescently labeled oligonucleotide structures and associated systems and methods are generally described. Some aspects of the disclosure are directed to a luminescently labeled oligonucleotide structure comprising a double-stranded oligonucleotide, where each strand is labeled with one or more types of luminescent label, and where a minimum distance between each type of luminescent label is relatively large (e.g., at least 10 nm). Some aspects of the disclosure are directed to a luminescently labeled oligonucleotide structure comprising a plurality of luminescently labeled double-stranded oligonucleotides connected by one or more binding molecules (e.g., multivalent proteins, such as avidin proteins). In certain embodiments, one or more luminescently labeled double-stranded oligonucleotides of the plurality of luminescently labeled double-stranded oligonucleotides comprise one or more isocytosine or isoguanine nucleotides, and one or more luminescently labeled double-stranded oligonucleotides of the plurality of luminescently labeled double-stranded oligonucleotides do not comprise any isocytosine or isoguanine nucleotides. Some aspects of the disclosure are directed to a set of luminescently labeled structures comprising one or more luminescently labeled oligonucleotide structures, where each structure of the set has one or more unique luminescence characteristics (e.g., lifetime, intensity).

A luminescent label generally refers to a molecule that absorbs one or more photons and may subsequently emit one or more photons after one or more time durations. In some embodiments, the term “luminescent label” is used interchangeably with “label” or “luminescent molecule.” Luminescent labels may be used in a variety of systems and methods for detecting and/or characterizing biological analytes, including but not limited to systems and methods for sequencing polypeptides and/or nucleic acids. In certain embodiments, these systems and methods may involve monitoring a biological reaction in real time using a plurality of types of luminescently labeled reaction components. As an illustrative example, a system or method for polypeptide sequencing may comprise a plurality of types of luminescently labeled amino acid recognition molecules, where each type of amino acid recognition molecule is labeled with a different type of luminescent label. As another illustrative example, a system or method for nucleic acid sequencing may comprise a plurality of types of luminescently labeled nucleotides, where each type of nucleotide (e.g., deoxyadenosine triphosphate (dATP), thymidine triphosphate (TTP), deoxyguanosine triphosphate (dGTP), deoxycytidine triphosphate (dCTP)) is labeled with a different type of luminescent label. In some embodiments, the luminescently labeled reaction components (e.g., amino acid recognition molecules, nucleotides) may be illuminated by a light source to cause luminescence, and the resulting luminescent light may be detected by one or more photodetectors. The detected luminescent light may be recorded and analyzed to identify or otherwise characterize the type of reaction component based on one or more luminescent properties of the detected luminescent light. In order to be able to identify or otherwise characterize the type of luminescently labeled reaction component emitting the detected luminescent light, each type of reaction component may be labeled with a luminescent label having readily differentiable luminescent properties (e.g., lifetime, intensity).

In some cases, a set of luminescent labels may comprise one or more luminescently labeled oligonucleotide structures described herein. In some cases, one or more luminescent properties of a luminescently labeled oligonucleotide may be tuned to be distinct from the luminescent properties of other luminescent labels in a set by attaching varying numbers and/or types of fluorophores to oligonucleotide strands. In some cases, this may advantageously allow for the development of luminescently labeled oligonucleotide structures having different luminescent properties from known fluorophores. In some cases, this may allow for the development of a set of luminescent labels having distinct values for one or more luminescent properties. In an illustrative, non-limiting embodiment, a set of luminescent labels may comprise a first known fluorophore (e.g., Cy®3), a second known fluorophore (e.g., Cy®3B), and a luminescently labeled oligonucleotide structure comprising a first oligonucleotide strand comprising one or more copies of the first known fluorophore and a second oligonucleotide strand comprising one or more copies of the second known fluorophore. In certain embodiments, one or more luminescent properties (e.g., lifetime, intensity) of the luminescently labeled oligonucleotide structure may differ from those of the first known fluorophore and those of the second known fluorophore. In some cases, the one or more luminescent properties of the luminescently labeled oligonucleotide structure may be varied by adding or removing copies of the first known fluorophore and/or the second known fluorophore. In certain instances, for example, luminescent intensity may be increased by adding additional copies of the first known fluorophore and/or the second known fluorophore.

Some aspects are directed to a set of two or more luminescent labels, where each luminescent label of the set has a value for one or more luminescent properties (e.g., lifetime, intensity) that differ from the values for other luminescent labels of the set by a certain minimum amount. In certain embodiments, a minimum percentage difference between values of one or more luminescent characteristics for any two labels of a set of two or more luminescent labels may be relatively large. In some instances, a set of luminescent labels comprises a plurality of luminescent labels, where each luminescent label has a different bin ratio. In certain instances, a minimum difference between the bin ratio values of any two luminescent labels of the set of luminescent labels is at least 0.1. In some instances, a set of luminescent labels comprises a plurality of luminescent labels, where each luminescent label occupies of a distinct spatial region of a two-dimensional plot of two luminescence characteristics (e.g., a plot of intensity v. bin ratio).

### Luminescently Labeled Oligonucleotide Structures

In some embodiments, assembling a plurality of pairs of hybridized oligonucleotide strands using one or more binding molecules may advantageously provide structures with large numbers of fluorophores while maintaining a sufficient distance between fluorophores to prevent energy transfer between fluorophores, which can decrease luminescence lifetime.

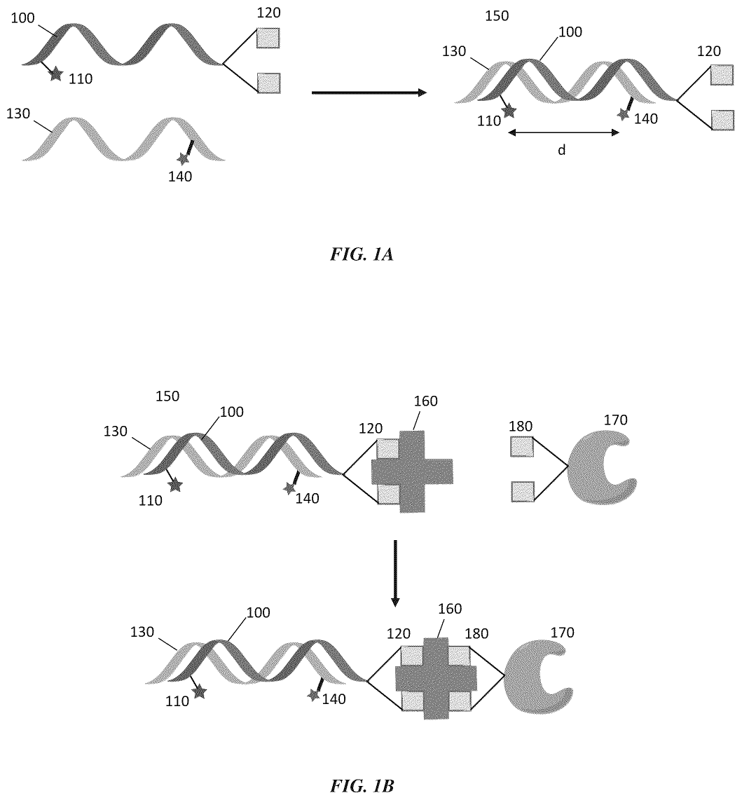

A schematic illustration of an exemplary luminescently labeled oligonucleotide structure is shown in FIG. 1A. In FIG. 1A, first single-stranded oligonucleotide 100 comprises one copy of first luminescent label 110. In certain embodiments, first single-stranded oligonucleotide 100 further comprises first binding moiety 120. In addition, first complementary single-stranded oligonucleotide 130 comprises one copy of second luminescent label 140. As shown in FIG. 1A, first single-stranded oligonucleotide 100 and first complementary single-stranded oligonucleotide 130 may be hybridized to form luminescently labeled oligonucleotide 150. In luminescently labeled oligonucleotide 150, first luminescent label 110 and second luminescent label 140 may be separated by a minimum distance d. In some embodiments, minimum distance d may be relatively large (e.g., at least 10 nm).

In some embodiments, a luminescently labeled oligonucleotide structure may be bound to a reaction component (e.g., an amino acid recognition molecule, a nucleotide) through a binding molecule. A schematic illustration of an exemplary reaction component labeled with a luminescently labeled oligonucleotide structure is shown in FIG. 1B. In FIG. 1B, luminescently labeled oligonucleotide structure 150 comprises first binding moiety 120, which is bound to first binding molecule 160. In some embodiments, reaction component 170 comprises second binding moiety 180. In some embodiments, second binding moiety 180 also binds to first binding molecule 160, thereby conjugating luminescently labeled oligonucleotide structure 150 to reaction component 170. In certain embodiments, first binding moiety 120 and second binding moiety 180 may each comprise a biotin moiety (e.g., a bis-biotin moiety), and first binding molecule 160 may comprise a multivalent protein, such as an avidin protein (e.g., a streptavidin protein).

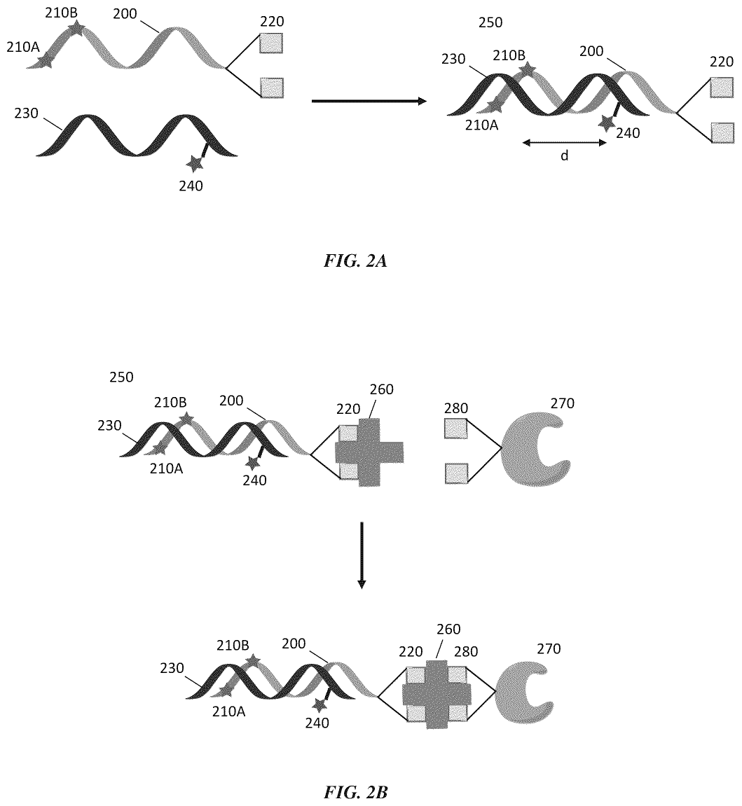

In some embodiments, a luminescently labeled oligonucleotide structure comprises a plurality of first luminescent labels and/or second luminescent labels. FIG. 2A shows a schematic illustration of an exemplary luminescently labeled oligonucleotide structure comprising two copies of a first luminescent label (also referred to as two first luminescent labels) and one copy of a second luminescent label (also referred to as one second luminescent label). In FIG. 2A, first single-stranded oligonucleotide 200 comprises two copies of first luminescent label 210: first copy 210A and second copy 210B. In certain embodiments, first single-stranded oligonucleotide 200 further comprises first binding moiety 220. In addition, first complementary single-stranded oligonucleotide 230 comprises one copy of second luminescent label 240. As shown in FIG. 2A, first single-stranded oligonucleotide 200 and first complementary single-stranded oligonucleotide 230 may be hybridized to form luminescently labeled oligonucleotide 250. In luminescently labeled oligonucleotide 250, a minimum distance d between any first luminescent label 210 and any second luminescent label 240 (i.e., between the second copy of the first luminescent label 210B and the first copy of the second luminescent label 240 in FIG. 2A) may be relatively large (e.g., at least 10 nm).

FIG. 2B shows a schematic illustration of an exemplary luminescently labeled oligonucleotide structure bound to a reaction component (e.g., an amino acid recognition molecule, a nucleotide) through a binding molecule. In FIG. 2B, luminescently labeled oligonucleotide structure 250 comprises first binding moiety 220, which is bound to first binding molecule 260. In some embodiments, reaction component 270 comprises second binding moiety 280. In some embodiments, second binding moiety 280 also binds to first binding molecule 260, thereby conjugating luminescently labeled oligonucleotide structure 250 to reaction component 270. In certain embodiments, first binding moiety 220 and second binding moiety 280 may each comprise a biotin moiety (e.g., a bis-biotin moiety), and first binding molecule 260 may comprise an avidin protein (e.g., a streptavidin protein).

In some embodiments, a first single-stranded oligonucleotide of a luminescently labeled oligonucleotide structure comprises one or more copies of a first luminescent label. In certain embodiments, the first single-stranded oligonucleotide comprises two or more copies of the first luminescent label, three or more copies of the first luminescent label, four or more copies of the first luminescent label, five or more copies of the first luminescent label, six or more copies of the first luminescent label, seven or more copies of the first luminescent label, eight or more copies of the first luminescent label, nine or more copies of the first luminescent label, or ten or more copies of the first luminescent label.

In some embodiments, the first single-stranded oligonucleotide comprises one or more luminescent labels that are different from the first luminescent label. In certain embodiments, the first single-stranded oligonucleotide comprises one or more copies of a third luminescent label, wherein the third luminescent label is different from the first luminescent label. In some instances, the first single-stranded oligonucleotide comprises two or more copies of the third luminescent label, three or more copies of the third luminescent label, four or more copies of the third luminescent label, five or more copies of the third luminescent label, six or more copies of the third luminescent label, seven or more copies of the third luminescent label, eight or more copies of the third luminescent label, nine or more copies of the third luminescent label, or ten or more copies of the third luminescent label. In certain embodiments, the first single-stranded oligonucleotide further comprises one or more copies of additional luminescent labels that are different from the first and third luminescent labels.

In some embodiments, a first complementary single-stranded oligonucleotide of a luminescently labeled oligonucleotide structure comprises one or more copies of a second luminescent label. In some instances, the second luminescent label is different from the first luminescent label. In some instances, the second luminescent label is the same as the first luminescent label. In certain embodiments, the first complementary single-stranded oligonucleotide comprises two or more copies of the second luminescent label, three or more copies of the second luminescent label, four or more copies of the second luminescent label, five or more copies of the second luminescent label, six or more copies of the second luminescent label, seven or more copies of the second luminescent label, eight or more copies of the second luminescent label, nine or more copies of the second luminescent label, or ten or more copies of the second luminescent label.

In some embodiments, the first complementary single-stranded oligonucleotide comprises one or more luminescent labels that are different from the second luminescent label. In certain embodiments, the first complementary single-stranded oligonucleotide comprises one or more copies of a fourth luminescent label, wherein the fourth luminescent label is different from the second luminescent label. In some instances, the first complementary single-stranded oligonucleotide comprises two or more copies of the fourth luminescent label, three or more copies of the fourth luminescent label, four or more copies of the fourth luminescent label, five or more copies of the fourth luminescent label, six or more copies of the fourth luminescent label, seven or more copies of the fourth luminescent label, eight or more copies of the fourth luminescent label, nine or more copies of the fourth luminescent label, or ten or more copies of the fourth luminescent label. In certain embodiments, the first single-stranded oligonucleotide further comprises one or more copies of additional luminescent labels that are different from the second and fourth luminescent labels.

In some embodiments, a luminescent label described herein (e.g., a first luminescent label, a second luminescent label, a third luminescent label, a fourth luminescent label) is a fluorescent label (e.g., comprises a fluorescent dye). In some embodiments, a luminescent label comprises a cyanine, rhodamine, boron-dipyrromethene (BODIPY), fluorescein, acridine, phenoxazine, coumarin, porphyrin, phthalocyanine, naphthalimide, pyrene, anthracene, naphthalene, naphthylamine, stilbene, indole, benzindole, oxazole, carbazole, thiazole, benzothiazole, benzoxazole, phenanthridine, quinoline, ethidium, benzamide, carbocyanine, salicylate, anthranilate, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-RO, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-05, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

In certain embodiments, a luminescent label (e.g., a first luminescent label, a second luminescent label, a third luminescent label, a fourth luminescent label) comprises Cy®3, Cy®3B, ATTO Rho6G (also referred to as ATRho6G), Chromis 530N, and/or Chromis530N-S (also referred to as C530NS). In some embodiments, C530NS has the structure:

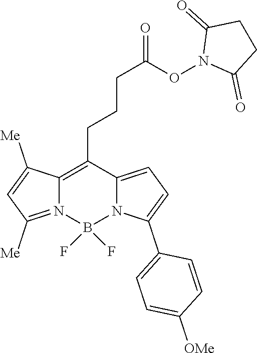

In some instances, the first single-stranded oligonucleotide of a luminescently labeled oligonucleotide structure comprises one first luminescent label comprising Cy®3B. In some instances, the first complementary single-stranded oligonucleotide comprises one second luminescent label comprising ATTO Rho6G.

In some instances, the first single-stranded oligonucleotide of a luminescently labeled oligonucleotide structure comprises one first luminescent label comprising ATTO Rho6G. In some instances, the first complementary single-stranded oligonucleotide comprises one second luminescent label comprising Cy®3B.

In some instances, the first single-stranded oligonucleotide of a luminescently labeled oligonucleotide structure comprises two first luminescent labels, each first luminescent label comprising Cy®3. In some instances, the first complementary single-stranded oligonucleotide comprises one second luminescent label comprising Cy®3B.

A luminescently labeled oligonucleotide structure may have any suitable length. In some embodiments, the luminescently labeled oligonucleotide structure has a length of at least 20 base pairs, at least 25 base pairs, at least 30 base pairs, at least 35 base pairs, at least 40 base pairs, at least 50 base pairs, at least 60 base pairs, at least 70 base pairs, at least 80 base pairs, at least 90 base pairs, or at least 100 base pairs. In some embodiments, the luminescently labeled oligonucleotide structure has a length in a range of 20-25 base pairs, 20-30 base pairs, 20-40 base pairs, 20-50 base pairs, 20-60 base pairs, 20-70 base pairs, 20-80 base pairs, 20-90 base pairs, 20-100 base pairs, 25-30 base pairs, 25-40 base pairs, 25-50 base pairs, 25-60 base pairs, 25-70 base pairs, 25-80 base pairs, 25-90 base pairs, 25-100 base pairs, 30-50 base pairs, 30-60 base pairs, 30-70 base pairs, 30-80 base pairs, 30-90 base pairs, 30-100 base pairs, 50-70 base pairs, 50-80 base pairs, 50-90 base pairs, 50-100 base pairs, 70-100 base pairs, 80-100 base pairs, or 90-100 base pairs.

Table 1 provides a list of example sequences of oligonucleotide strands of luminescently labeled oligonucleotide structures. It should be appreciated that these sequences and other examples described herein are meant to be non-limiting.

TABLE 1
Non-limiting examples of oligonucleotide sequences

| Oligo- | | |
|---|---|---|
| nucleotide | Name | Sequence |
| ODN1 | A | CGGATTTATTCATAGCTTGTGCTATGTGGCA |
| | | TCGATA/X/TAAGCG, where /X/ is |
| | | Cy®3B (SEQ ID NO: 1) |
| ODN2 | B | CGCTTATTATCGATGCCACATAGCACAAGCT |
| | | ATGAAT/Y/AATCCG, where /Y/ is |
| | | ATRho6G (SEQ ID NO: 2) |
| ODN1 | C | GGCTATTTATGTATGAGTTCATGTGATGCGA |
| | | GCTATAT/X/TAGGCAT/X/TACGG, |
| | | where /X/ is Cy®3 (SEQ ID NO: 3) |
| ODN2 | D | CCGTTGCCTTATAGCTCGCATCACATGAACT |
| | | CATACATA/Y/ATAGCC, where /Y/ is |
| | | Cy®3B (SEQ ID NO: 4) |
| ODN1 | E | AGGCGT/10/TGCACGT/10/TGCCGTTGCC |
| | | TCGACAGATCCCGA, where /10/ is |
| | | Cy®3 (SEQ ID NO: 5) |
| ODN2 | F | TCGGT/10/TGATCTGTCGAGT/10/TGCAA |
| | | CGGCCGTGCCGCCT, where /10/ is |
| | | Cy®3 (SEQ ID NOs: 6 and 7, from |
| | | left to right) |

In some embodiments, one or more oligonucleotide strands of a luminescently labeled oligonucleotide structure have a sequence that has at least 50%, at least 60%, at least 70%, at least 80%, at least 90%, at least 95%, at least 99%, or 100% sequence identity to a sequence selected from Tables 1-3. In some embodiments, one or more oligonucleotide strands of a luminescently labeled oligonucleotide structure have 25-50%, 50-60%, 60-70%, 70-80%, 80-90%, 90-95%, or 95-99%, or higher, sequence identity to a sequence listed in Tables 1-3. In some embodiments, an oligonucleotide strand includes one or more nucleotide deletions, additions, or mutations relative to a sequence set forth in Tables 1-3. In some embodiments, an oligonucleotide strand includes a deletion, addition, or mutation of 1, 2, 3, 4, 5, 6, 10, 20, 50, or more nucleotides (which may or may not be consecutive nucleotides) relative to a sequence set forth in Tables 1-3.

In some embodiments, different types of labels are separated by a certain minimum distance. Without wishing to be bound by any particular theory, separation by a certain minimum distance may advantageously prevent energy transfer between a first type of luminescent label and a second type of luminescent label. In some cases, separation by a certain minimum distance may advantageously prevent Förster resonance energy transfer (FRET).

In some embodiments, a minimum distance between any first luminescent label and any second luminescent label is at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 25 nm, at least 30 nm, at least 35 nm, at least 40 nm, or at least 50 nm. In some embodiments, a minimum distance between any two luminescent labels can be approximated as 0.34*n, where n is the number of nucleotide bases between the luminescent labels. In some cases, a minimum distance between two luminescent labels can be measured as the distance between the geometric centers of the luminescent labels. A geometric center of a molecule, in some embodiments, refers to the average position of all atoms of the molecule (e.g., all atoms in a luminescent label), wherein the atoms are not weighted. Thus, in some embodiments, the geometric center of a molecule refers to a point in space that is an average of the coordinates of all atoms in the molecule. In some embodiments, the minimum distance d can be obtained, for example, using theoretical methods known in the art (e.g., computationally or otherwise). In some embodiments, theoretical methods can include any approach that accounts for molecular structure, such as bond lengths, bond angles and rotation, electrostatics, nucleic acid helicity, and other physical factors which might be representative of a molecule in solution. In some embodiments, distance measurements can be obtained experimentally, e.g., by crystallographic or spectroscopic means.

In some embodiments, a minimum distance between attachment sites of luminescent labels to oligonucleotide strands of the luminescently labeled oligonucleotide structure may be relatively large. In some cases, a distance between attachment sites of luminescent labels to oligonucleotide strands can be described by the number of intervening unlabeled nucleotides (e.g., intervening bases). It should be understood that the number of nucleotides can refer to either the number of nucleotide bases in a single-stranded nucleic acid or the number of nucleotide base pairs in a double-stranded nucleic acid. In some embodiments, a minimum distance between an attachment site of any first luminescent label and an attachment site of any second luminescent label is at least 5, at least 10, at least 15, at least 20, at least 25, at least 30, at least 35, at least 40, at least 45, least 50, or at least 100 unlabeled nucleotides. In some embodiments, a minimum distance between an attachment site of any first luminescent label and an attachment site of any second luminescent label is between 5 and 10, 5 and 20, 4 and 30, 5 and 40, 5 and 50, 5 and 100, 10 and 20, 10 and 30, 10 and 40, 10 and 50, 10 and 100, 20 and 30, 20 and 40, 20 and 50, 20 and 100, 30 and 50, 30 and 100, and 50 and 100 unlabeled nucleotides.

In some embodiments, one or more oligonucleotide strands of a luminescently labeled oligonucleotide structure comprises a binding moiety. In certain embodiments, the first single-stranded oligonucleotide comprises a binding moiety. In certain embodiments, the first complementary single-stranded oligonucleotide comprises a binding moiety.

In some embodiments, the binding moiety comprises at least one biotin moiety. In certain embodiments, the at least one biotin moiety comprises a bis-biotin moiety. In some embodiments, the binding group further comprises a tag sequence. In some embodiments, a tag sequence comprises at least one biotin ligase recognition sequence that permits biotinylation of the linker (e.g., incorporation of one or more biotin moieties, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In some embodiments, the binding group of the linker comprises at least one biotin ligase recognition sequence having the biotin moiety attached thereto or at least two biotin ligase recognition sequences having the biotin moiety attached thereto.

In some embodiments, the binding moiety of the luminescently labeled oligonucleotide structure comprises or is conjugated to a binding molecule. In some embodiments, the first binding molecule comprises a multivalent protein (e.g., a protein having more than one ligand binding site that can independently bind a ligand). In some embodiments, the first binding molecule comprises an avidin protein. The term “avidin protein” refers to a biotin-binding protein, generally having a biotin binding site at each of four subunits of the avidin protein. Avidin proteins include, for example, avidin, streptavidin, traptavidin, tamavidin, bradavidin, xenavidin, and homologs and variants thereof. In certain embodiments, the avidin protein comprises streptavidin. In certain embodiments, the avidin protein is in a monomeric, dimeric, or tetrameric form. In some embodiments, the avidin protein is streptavidin in a tetrameric form (e.g., a homotetramer).

In some embodiments, the binding moiety comprises a click chemistry handle. The term “click chemistry handle,” as used herein, refers to a reactant, or a reactive group, that can partake in a click chemistry reaction. For example, a strained alkyne, e.g., a cyclooctyne, is a click chemistry handle since it can partake in a strain-promoted cycloaddition. In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Additional suitable click chemistry handles are well known to those of skill in the art, and such click chemistry handles include, but are not limited to, the click chemistry reaction partners, groups, and handles described in Becer, Hoogenboom, and Schubert, Click Chemistry beyond Metal-Catalyzed Cycloaddition, Angewandte Chemie International Edition (2009) 48: 4900-4908 and PCT/US2012/044584 and references therein, which references are incorporated herein by reference for click chemistry handles and methodology.

In some embodiments, the first binding molecule may be used to form a covalent or non-covalent linkage between a luminescently labeled oligonucleotide structure and one or more reaction components (e.g., amino acid recognition molecule, aminopeptidase, nucleotide). In certain embodiments, the first binding molecule may be bound to an amino acid recognition molecule. In certain embodiments, the first binding molecule may be bound to an aminopeptidase. In certain embodiments, the first binding molecule may be bound to a nucleotide.

### Multi-Oligonucleotide Structures

Some embodiments are directed to a luminescently labeled oligonucleotide structure comprising multiple oligonucleotide strands assembled through one or more binding molecules (e.g., through biotin/streptavidin conjugation). For example, some embodiments are directed to a luminescently labeled oligonucleotide structure comprising a first single-stranded, biotinylated oligonucleotide bound to a first streptavidin; a first complementary single-stranded oligonucleotide hybridized to the first single-stranded, biotinylated oligonucleotide; a second single-stranded, biotinylated oligonucleotide bound to the first streptavidin; a second complementary single-stranded oligonucleotide hybridized to the second single-stranded, biotinylated oligonucleotide, wherein the second complementary single-stranded oligonucleotide is biotinylated and bound to a second streptavidin; and at least one luminescent label bound to at least one single-stranded oligonucleotide.

In some embodiments, two or more pairs of oligonucleotides separated by one or more binding molecules (e.g., an avidin protein) have sequences formed using different systems of nucleotides. In certain embodiments, a first pair of oligonucleotides (e.g., a first single-stranded oligonucleotide and a first complementary single-stranded oligonucleotide) comprises sequences consisting of four types of nucleotides: A, C, G, and/or T. In some cases, oligonucleotides comprising sequences consisting of A, C, G, and/or T may be referred to as “GCAT system oligonucleotides.” In certain embodiments, a second pair of nucleotides (e.g., a second single-stranded oligonucleotide and a second complementary single-stranded oligonucleotide) comprises sequence formed from at least six types of nucleotides: A, C, G, T, isoguanine (iG), and isocytosine (iC). In some cases, oligonucleotides comprising sequences formed from at least A, C, G, T, iG, and/or iC may be referred to as a “GCATiGiC system oligonucleotide.” In some cases, utilization of two or more systems of nucleotides may advantageously facilitate assembly of multiple-oligonucleotide structures. In certain cases, for example, use of two or more systems of nucleotides may advantageously enhance orthogonality and may reduce luminescently labeled single-stranded oligonucleotides hybridizing to the incorrect strands.

In some embodiments, a luminescently labeled oligonucleotide comprises adenine and thymine base pairs. In some embodiments, a luminescently labeled oligonucleotide comprises guanine and cytosine base pairs. In some embodiments, a luminescently labeled oligonucleotide comprises isoguanine and isocytosine base pairs (iG:iC base pair). In some embodiments, a luminescently labeled oligonucleotide comprises 2,6-diaminopurine (diamino purine) and thymine nucleotide base pairs.

In some embodiments, isoguanine has the structure:

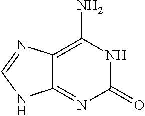 [SMILES: Nc1[nH]c(=O)nc2[nH]cnc12]

In some embodiments, isocytosine has the structure:

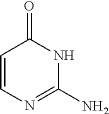

In some embodiments, diaminopurine has the structure:

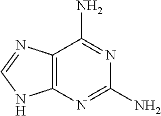 [SMILES: Nc1nc(N)c2nc[nH]c2n1]

In some embodiments, the first single-stranded, biotinylated oligonucleotide comprises an isoguanine and/or an isocytosine and the first complementary single-stranded oligonucleotide comprises an isocytosine and/or an isoguanine, or wherein the second single-stranded, biotinylated oligonucleotide comprises an isoguanine and/or an isocytosine and the second complementary single-stranded oligonucleotide comprises an isocytosine and/or an isoguanine. In some embodiments, the oligonucleotide structure further comprises a dye-labeled nucleoside or amino acid recognition molecule bound to the second streptavidin. In some embodiments, the first complementary single-stranded oligonucleotide is bound to a terminator.

Certain aspects of the disclosure relate to a method of assembling a luminescently labeled oligonucleotide structure described herein comprising contacting a first single-stranded, biotinylated oligonucleotide with a first streptavidin; contacting a second single-stranded, biotinylated oligonucleotide with the first streptavidin; contacting the first single-stranded, biotinylated oligonucleotide with a first complementary single-stranded oligonucleotide; and contacting the second single-stranded, biotinylated oligonucleotide with a second complementary single-stranded oligonucleotide. In some embodiments, at least one of the first single-stranded, biotinylated oligonucleotide, first complementary single-stranded oligonucleotide, second single-stranded, biotinylated oligonucleotide, and second complementary single-stranded oligonucleotide comprises at least one luminescent label. In some embodiments, the first single-stranded, biotinylated oligonucleotide comprises an isoguanine and/or an isocytosine and the first complementary single-stranded oligonucleotide comprises an isocytosine and/or an isoguanine, or wherein the second single-stranded, biotinylated oligonucleotide comprises an isoguanine and/or an isocytosine and the second complementary single-stranded oligonucleotide comprises an isocytosine and/or an isoguanine. In some embodiments, the first complementary single-stranded oligonucleotide is biotinylated. In some embodiments, the first complementary single-stranded oligonucleotide is luminescently labeled. In some embodiments, the second complementary single-stranded oligonucleotide is luminescently labeled. In some embodiments, the method is repeated one or two times.

In some embodiments, methods provided herein comprise assembling a luminescently labeled oligonucleotide structure comprising multiple luminescently labeled oligonucleotides. In some embodiments, a luminescently labeled oligonucleotide is limited in the number of dyes that can be bound to the oligonucleotide. In some embodiments, the limitation is due to dye-dye interactions. The present disclosure relates to the discovery that this limitation can be overcome by conjugating multiple luminescently labeled oligonucleotides together, rather than adding additional dyes to the same oligonucleotide. The present disclosure also relates to the discovery that the length of the oligonucleotide structure is limited due to oligonucleotide bending or curving, that is, as more oligonucleotides are added to the luminescently labeled oligonucleotide structure. The present disclosure relates to the discovery that the incorporation additional nucleotide bases (i.e., isoguanine and isocytosine, in addition to adenine, guanine, cytosine, and thymine) facilitates the conjugation of several luminescently labeled oligonucleotides without the limitation of oligonucleotide bending or curving.

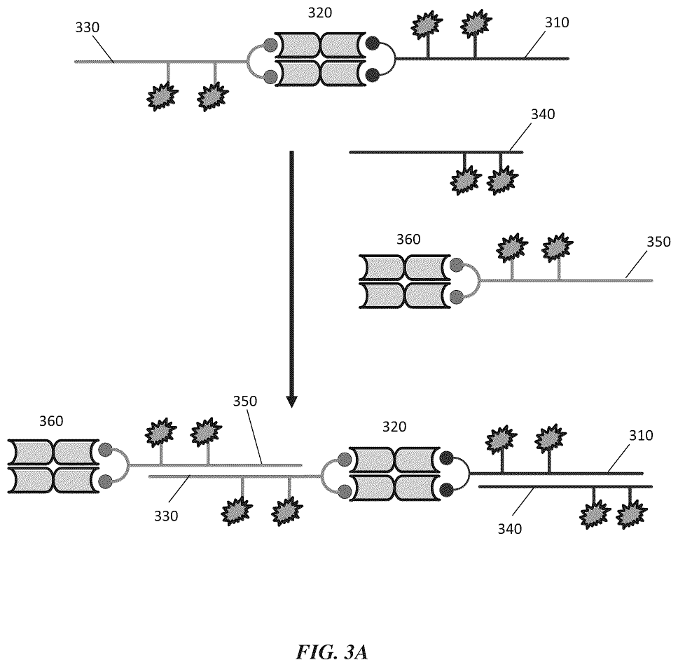

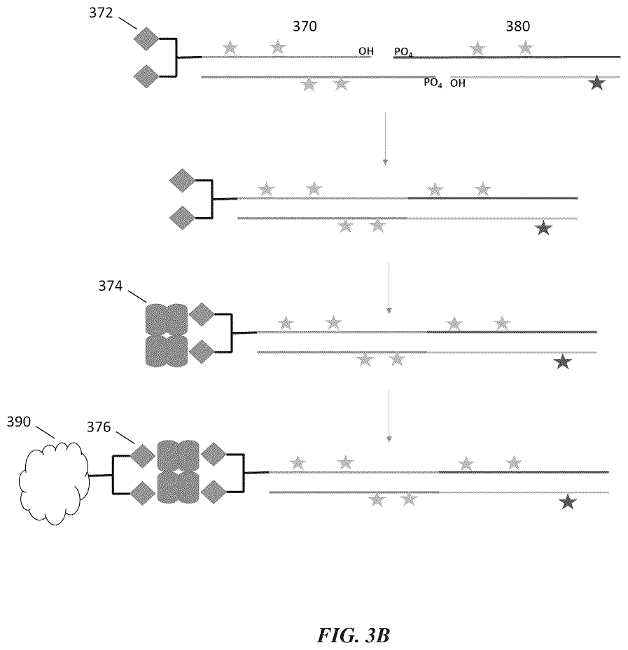

FIG. 3A shows a schematic illustration of an exemplary method of assembling a luminescently labeled oligonucleotide structure, according to some embodiments. In some embodiments, assembly of the luminescently labeled oligonucleotide structure begins with first biotinylated, luminescently labeled oligonucleotide strand 310. In certain embodiments, strand 310 has a sequence consisting of four different types of nucleotide bases. In some embodiments, strand 310 is conjugated to streptavidin 320. In some embodiments, a second biotinylated single-stranded, luminescently labeled oligonucleotide 330 is conjugated to streptavidin 320. In some embodiments, strand 330 has a sequence comprising six different types of nucleotide bases. In some embodiments, a first complementary luminescently labeled oligonucleotide 340 is hybridized to strand 310. Like strand 310, strand 340 has a sequence comprising only four different types of nucleotide bases. In some embodiments, a second complementary luminescently labeled oligonucleotide 350 is hybridized to strand 330. Like strand 330, strand 350 has a sequence comprising six different types of nucleotide bases. In some embodiments, strand 350 is biotinylated. In some embodiments, strand 350 is conjugated to second streptavidin 360. A fully assembled oligonucleotide structure is shown in FIG. 3A.

In some embodiments, the luminescently labeled oligonucleotide structure described herein comprises at least one, at least two, at least three, at least four, at least five, at least six, at least seven, or at least eight luminescent labels. In some embodiments, additional biotinylated luminescently labeled oligonucleotides can be added to the luminescently labeled oligonucleotide structure to increase the number of luminescent labels. In some embodiments, an amino acid recognition molecule can be added to the end of the luminescently labeled oligonucleotide structure for use in polypeptide sequencing. In some embodiments, a nucleotide can be added to the end of the luminescently labeled oligonucleotide structure for use in nucleic acid sequencing.

In some embodiments, the luminescently labeled oligonucleotide structure may have any suitable length. In some embodiments, the luminescently labeled oligonucleotide structure has a length of at least 20 base pairs, at least 25 base pairs, at least 30 base pairs, at least 35 base pairs, at least 40 base pairs, at least 50 base pairs, at least 60 base pairs, at least 70 base pairs, at least 80 base pairs, at least 90 base pairs, or at least 100 base pairs. In some embodiments, the luminescently labeled oligonucleotide structure has a length in a range of 20-25 base pairs, 20-30 base pairs, 20-40 base pairs, 20-50 base pairs, 20-60 base pairs, 20-70 base pairs, 20-80 base pairs, 20-90 base pairs, 20-100 base pairs, 25-30 base pairs, 25-40 base pairs, 25-50 base pairs, 25-60 base pairs, 25-70 base pairs, 25-80 base pairs, 25-90 base pairs, 25-100 base pairs, 30-50 base pairs, 30-60 base pairs, 30-70 base pairs, 30-80 base pairs, 30-90 base pairs, 30-100 base pairs, 50-70 base pairs, 50-80 base pairs, 50-90 base pairs, 50-100 base pairs, 70-100 base pairs, 80-100 base pairs, or 90-100 base pairs.

In some embodiments, the at least one luminescent label is fluorescent (e.g., comprises a fluorophore). The at least one luminescent label may be any luminescent label described herein. In certain embodiments, the at least one luminescent label comprises Cy®3, Cy®3B, ATRho6G, Chromis 530N, and/or C530NS.

In some embodiments, any single-stranded oligonucleotide comprising a luminescent label comprises one, two, three, or four luminescent labels. In some embodiments, the oligonucleotide structure comprises at least four luminescent labels or at least eight luminescent labels.

In some embodiments, the luminescently labeled oligonucleotide structure further comprises a third single-stranded, biotinylated oligonucleotide bound to the second streptavidin. In some embodiments, the oligonucleotide structure further comprises a third complementary single-stranded oligonucleotide hybridized to the third single-stranded, biotinylated oligonucleotide, wherein the third complementary single-stranded oligonucleotide is biotinylated and bound to a third streptavidin. In some embodiments, the dye-labeled nucleoside or amino acid recognition molecule is bound to the third streptavidin. In some embodiments, the oligonucleotide structure further comprises a fourth single-stranded, biotinylated oligonucleotide bound to the third streptavidin. In some embodiments, the oligonucleotide structure further comprises a fourth complementary single-stranded oligonucleotide hybridized to the fourth single-stranded, biotinylated oligonucleotide, wherein the fourth complementary single-stranded oligonucleotide is biotinylated and bound to a fourth streptavidin. In some embodiments, the dye-labeled nucleoside or amino acid recognition molecule is bound to the fourth streptavidin.

In some embodiments, the second complementary single-stranded oligonucleotide is bound to a second binding molecule. In some embodiments, a third single-stranded oligonucleotide is bound to the second binding molecule. In some embodiments, a third complementary single-stranded oligonucleotide is hybridized to the third single-stranded oligonucleotide. In certain embodiments, the second binding molecule comprises an avidin protein. In certain instances, the avidin protein comprises streptavidin.

Aspects of the disclosure relate to a system comprising a chip comprising a plurality of wells, wherein one or more wells of the plurality of wells are adapted to receive a peptide and have the peptide bound to a surface thereof; and a dye-labeled nucleoside or amino acid recognition molecule bound to a luminescently labeled oligonucleotide described herein. In some embodiments, the dye-labeled nucleoside or amino acid recognition molecule is configured to bind to a terminal nucleotide of the nucleic acid or a terminal amino acid of the peptide. In some embodiments, the plurality of wells comprises 96 wells, 384 wells, 1,536 wells, or more wells. In some embodiments, the peptide is derived from a sample comprising a plurality of peptides. In some embodiments, the peptide is immobilized to the base of a well of the plurality of wells via a secondary complex. In some embodiments, the secondary complex is a streptavidin-biotin complex.

Aspects of the disclosure relate to methods of nucleotide and/or polypeptide sequencing comprising contacting a single nucleic acid or polypeptide molecule with one or more dye-labeled nucleosides or amino acid recognition molecules bound to a structure described herein; and detecting a series of signal pulses indicative of association of the one or more dye-labeled nucleoside or amino acid recognition molecules with successive nucleotides or amino acids exposed at a terminus of the single nucleic acid or polypeptide while the single nucleic acid or polypeptide is being synthesized or degraded, thereby sequencing the single nucleic acid or polypeptide molecule. In some embodiments, association of the one or more structures with each type of nucleotide or amino acid exposed at the terminus produces a characteristic pattern in the series of signal pulses that is different from other types of nucleotides or amino acids exposed at the terminus. In some embodiments, the characteristic pattern comprises a portion of the series of signal pulses. In some embodiments, a signal pulse of the characteristic pattern corresponds to an individual association event between a dye-labeled nucleoside or amino acid recognition molecule and a nucleotide or amino acid exposed at the terminus. In some embodiments, the signal pulse of the characteristic pattern comprises a pulse duration that is characteristic of a dissociation rate of binding between the dye-labeled nucleoside or amino acid recognition molecule and the nucleotide or amino acid exposed at the terminus. In some embodiments, each signal pulse of the characteristic pattern is separated from another by an interpulse duration that is characteristic of an association rate of dye-labeled nucleoside or amino acid recognition molecule binding. In some embodiments, the characteristic pattern corresponds to a series of reversible dye-labeled nucleoside or amino acid recognition molecule binding interactions with the nucleotide or amino acid exposed at the terminus of the single polypeptide molecule. In some embodiments, the series of reversible dye-labeled nucleoside or amino acid recognition molecule binding interactions comprises a reversible formation of one binary complex species at the terminus of the single polypeptide molecule. In some embodiments, wherein the series of reversible dye-labeled nucleoside or amino acid recognition molecule binding interactions comprises a reversible formation of different binary complex species at the terminus of the single polypeptide molecule. In some embodiments, the characteristic pattern is indicative of the nucleotide or amino acid exposed at the terminus of the single polypeptide molecule and a nucleotide or amino acid at a contiguous position. In some embodiments, the nucleotide or amino acid exposed at the terminus and the nucleotide or amino acid at the contiguous position are of a different type. In some embodiments, sequencing comprises identifying each type of successive nucleotide or amino acid exposed at the terminus of the single polypeptide while the single nucleic acid polypeptide is being synthesized or degraded. In some embodiments, sequencing comprises identifying a portion of all types of successive nucleotides or amino acids exposed at the terminus of the single polypeptide while the single polypeptide is being synthesized or degraded. In some embodiments, sequencing comprises determining the relative positions of successive nucleotide or amino acid exposed at the terminus of the single nucleic acid or polypeptide while the single nucleic acid or polypeptide is being synthesized or degraded. In some embodiments, sequencing comprises identifying at least two contiguous nucleotides or amino acids in the single nucleic acid or polypeptide molecule. In some embodiments, sequencing comprises identifying at least two non-contiguous nucleotides or amino acids in the single nucleic acid or polypeptide molecule.

### Ligated Oligonucleotide Structures

Some embodiments are directed to a luminescently labeled oligonucleotide structure comprising multiple oligonucleotide strands assembled by ligation, and methods of preparing the same. For example, some embodiments relate to methods of preparing a luminescently labeled reaction component by ligating the ends of one double-stranded oligonucleotide to the ends of another double-stranded oligonucleotide, where each double-stranded oligonucleotide comprises one or more luminescent labels described herein.

FIG. 3B shows a schematic illustration of an example method of assembling a luminescently labeled oligonucleotide structure by ligation, according to some embodiments. In some embodiments, a first double-stranded oligonucleotide 370 is provided, where one or both strands of first double-stranded oligonucleotide 370 comprise one or more luminescent labels. In some embodiments, one strand of first double-stranded oligonucleotide 370 comprises a first binding moiety (e.g., a first biotin moiety, such as a first bis-biotin moiety). In some embodiments, a second double-stranded oligonucleotide 380 is provided, where one or both strands of second double-stranded oligonucleotide 380 comprise one or more luminescent labels.

In some embodiments, first double-stranded oligonucleotide 370 and/or second double-stranded oligonucleotide 380 comprise structures according to the luminescently labeled oligonucleotide or multi-oligonucleotide structures as described herein. For example, in some embodiments, the one or more luminescent labels of a first and/or second double-stranded oligonucleotide are separated from one another by a distance of at least 10 nm. In some embodiments, the first double-stranded oligonucleotide comprises one or more isoguanine and/or isocytosine nucleotides, and the second double-stranded oligonucleotide does not comprise one or more isoguanine and/or isocytosine nucleotides. In some embodiments, the second double-stranded oligonucleotide comprises one or more isoguanine and/or isocytosine nucleotides, and the first double-stranded oligonucleotide does not comprise one or more isoguanine and/or isocytosine nucleotides. In some embodiments, the first or second double-stranded oligonucleotide comprises at least one diaminopurine nucleotide.

In some embodiments, one or more oligonucleotide strands of first double-stranded oligonucleotide 370 and/or second double-stranded oligonucleotide 380 have a sequence that has at least 50%, at least 60%, at least 70%, at least 80%, at least 90%, at least 95%, at least 99%, or 100% sequence identity to a sequence selected from Tables 1-3. In some embodiments, one or more oligonucleotide strands of first double-stranded oligonucleotide 370 and/or second double-stranded oligonucleotide 380 have 25-50%, 50-60%, 60-70%, 70-80%, 80-90%, 90-95%, or 95-99%, or higher, sequence identity to a sequence listed in Tables 1-3. In some embodiments, an oligonucleotide strand includes one or more nucleotide deletions, additions, or mutations relative to a sequence set forth in Tables 1-3. In some embodiments, an oligonucleotide strand includes a deletion, addition, or mutation of 1, 2, 3, 4, 5, 6, 10, 20, 50, or more nucleotides (which may or may not be consecutive nucleotides) relative to a sequence set forth in Tables 1-3.

In some embodiments, first double-stranded oligonucleotide 370 and second double-stranded oligonucleotide 380 comprise complementary overhangs suitable for overhang ligation. For example, as generally depicted in FIG. 3B, each double-stranded oligonucleotide can comprise a double-stranded portion (e.g., a duplex portion) and a single-stranded portion (e.g., an unpaired portion), where the single-stranded portion forms an overhang. In some embodiments, the overhang is a 5′ overhang formed by a 5′ portion of one strand in each double-stranded oligonucleotide. In some embodiments, the overhang is a 3′ overhang formed by a 3′ portion of one strand in each double-stranded oligonucleotide. In some embodiments, the overhang comprises a phosphate (e.g., monophosphate). In some embodiments, the overhang is a 5′ overhang comprising a 5′-monophosphate. In some embodiments, first double-stranded oligonucleotide 370 comprises a first overhang, and second double-stranded oligonucleotide 380 comprises a second overhang that is complementary to the first overhang.

In some embodiments, first double-stranded oligonucleotide 370 comprising a first overhang is contacted with second double-stranded oligonucleotide 380 comprising a second overhang under hybridization conditions. In some embodiments, the hybridization conditions are sufficient to hybridize the first overhang of the first double-stranded oligonucleotide to the second overhang of the second double-stranded oligonucleotide. In some embodiments, the second overhang is fully complementary to the first overhang. However, full complementarity is not required, and in some embodiments, the second overhang is partially complementary to the first overhang, provided that the complementarity is sufficient for hybridizing the first and second overhangs under hybridization conditions.

In some embodiments, assembly of the luminescently labeled oligonucleotide structure proceeds by ligating first double-stranded oligonucleotide 370 to second double-stranded oligonucleotide 380. In some embodiments, ligating comprises enzymatic ligation. For example, in some embodiments, ligating comprises contacting the first and second double-stranded oligonucleotides with a ligase under ligation conditions. In some embodiments, the ligase is a DNA ligase (e.g., T4 DNA ligase). In some embodiments, the ligating comprises ligating both strands of first double-stranded oligonucleotide 370 to both strands of second double-stranded oligonucleotide 380. In some embodiments, the first overhang comprises a 5′-phosphate that is ligated to a 3′-hydroxyl of one strand of second double-stranded oligonucleotide 380, and the second overhang comprises a 5′-phosphate that is ligated to a 3′-hydroxyl of one strand of first double-stranded oligonucleotide 370.

In some embodiments, assembly of the luminescently labeled oligonucleotide structure proceeds by contacting the ligated first and second double-stranded oligonucleotides with a multivalent protein 374 that binds first binding moiety 372 to form a complex comprising the ligated double-stranded oligonucleotides and the multivalent protein. In some embodiments, multivalent protein 374 comprises an avidin protein (e.g., streptavidin), and first binding moiety 372 comprises a biotin moiety as described herein.

In some embodiments, assembly of the luminescently labeled oligonucleotide structure proceeds by contacting the complex with a reaction component 390 (e.g., an amino acid recognition molecule, a nucleotide) that comprises a second binding moiety 376, where multivalent protein 374 binds the second binding moiety to form a luminescently labeled reaction component.

### Sets of Luminescent Labels

Some aspects are directed to a set of luminescent labels comprising a plurality of luminescent labels. In some embodiments, each luminescent label of the set of luminescent labels has a distinct value for one or more luminescent characteristics. In some cases, a set of luminescent labels may advantageously be used to label a set of reaction components (e.g., amino acid recognition molecules) to ensure that each type of reaction component can be identified during protein sequencing and/or nucleic acid sequencing. In some embodiments, the set of luminescent labels may comprise one or more luminescently labeled oligonucleotide structures as described herein. In some embodiments, the set of luminescent labels may comprise one or more fluorophores known in the art (e.g., Cy®3, Cy®3B, ATTO Rho6G).

Non-limiting examples of luminescent characteristics include luminescent lifetime, luminescent intensity, bin ratio, and luminescent wavelength. In certain embodiments, each luminescent label has a value for a luminescent characteristic that differs from the value for the luminescent characteristic of each other luminescent label of the set of luminescent labels. In certain embodiments, a minimum percentage difference between luminescent characteristic values for any two luminescent labels of a set of luminescent labels is at least 1%, at least 5%, at least 10%, at least 20%, at least 30%, at least 50%, at least 100%, at least 150%, at least 200%, or at least 500%. In certain embodiments, a minimum percentage difference between luminescent characteristic values for any two luminescent labels of a set of luminescent labels is in a range from 1-5%, 1-10%, 1-20%, 1-30%, 1-50%, 1-100%, 1-150%, 1-200%, 1-500%, 5-10%, 5-20%, 5-30%, 5-50%, 5-100%, 5-150%, 5-200%, 5-500%, 10-20%, 10-30%, 10-50%, 10-100%, 10-150%, 10-200%, 10-500%, 20-50%, 20-100%, 20-150%, 20-200%, 20-500%, 50-100%, 50-150%, 50-200%, 50-500%, 100-200%, 100-500%, or 200-500%.

A set of luminescent labels may have any suitable number of luminescent labels. In certain embodiments, the set of luminescent labels comprises two or more luminescent labels, three or more luminescent labels four or more luminescent labels, four or more luminescent labels, five or more luminescent labels, six or more luminescent labels, seven or more luminescent labels, eight or more luminescent labels, nine or more luminescent labels, or ten or more luminescent labels. In some embodiments, the set of luminescent labels comprises two, three, four, five, six, seven, eight, nine, or ten luminescent labels, or more.

In some embodiments, the luminescent characteristic comprises a bin ratio. In certain cases, bin ratio may be a measurement of luminescent lifetime. In some cases, the bin ratio of a luminescent label may be obtained using an integrated device described herein. In some embodiments, the bin ratio of a luminescent label may refer to a ratio of photoelectrons collected during a first time period (bin 0) to photoelectrons collected during a second time period (bin 1). In certain embodiments, the first time period may start a relatively long time after an excitation pulse (e.g., 3 ns after an excitation pulse). In certain embodiments, the second time period may start a relatively short time after an excitation pulse (e.g., 1 ns after an excitation pulse). In some cases, a relatively low bin ratio may indicate that a dye has a relatively short luminescent lifetime. In some cases, a relatively high bin ratio may indicate that a dye has a relatively long luminescent lifetime.

In some embodiments, each luminescent label of a set of luminescent labels may have a distinct bin ratio value. In certain embodiments, a minimum difference between bin ratio values of a set of luminescent labels is at least 0.05, at least 0.1, at least 0.2, at least 0.3, at least 0.4, at least 0.5, at least 0.6, at least 0.7, at least 0.8, at least 0.9, or at least 1.0. In certain embodiments, a minimum difference between bin ratio values of a set of luminescent labels is in a range from 0.05 to 0.2, 0.05 to 0.3, 0.05 to 0.4, 0.05 to 0.5, 0.05 to 0.6, 0.05 to 0.7, 0.05 to 0.8, 0.05 to 0.9, 0.05 to 1.0, 0.1 to 0.2, 0.1 to 0.3, 0.1 to 0.4, 0.1 to 0.5, 0.1 to 0.6, 0.1 to 0.7, 0.1 to 0.8, 0.1 to 0.9, 0.1 to 1.0, 0.2 to 0.5, 0.2 to 0.6, 0.2 to 0.7, 0.2 to 0.8, 0.2 to 0.9, 0.2 to 1.0, 0.5 to 1.0, 0.6 to 1.0, 0.7 to 1.0, 0.8 to 1.0, or 0.9 to 1.0. In certain embodiments, a minimum percentage difference between bin ratio values of a set of luminescent labels is at least 1%, at least 5%, at least 10%, at least 20%, at least 30%, at least 50%, at least 100%, at least 150%, at least 200%, or at least 500%. In certain embodiments, a minimum percentage difference between bin ratio values of a set of luminescent labels is in a range from 1-5%, 1-10%, 1-20%, 1-30%, 1-50%, 1-100%, 1-150%, 1-200%, 1-500%, 5-10%, 5-20%, 5-30%, 5-50%, 5-100%, 5-150%, 5-200%, 5-500%, 10-20%, 10-30%, 10-50%, 10-100%, 10-150%, 10-200%, 10-500%, 20-50%, 20-100%, 20-150%, 20-200%, 20-500%, 50-100%, 50-150%, 50-200%, 50-500%, 100-200%, 100-500%, or 200-500%.

In some embodiments, each luminescent label of a set of luminescent labels has a unique combination of two or more different luminescence characteristics. In some embodiments, a system comprises a first luminescent label having a first ordered pair of characteristics comprising a first value of a first characteristic and a first value of a second characteristic. In some embodiments, a system comprises a second luminescent label having a second ordered pair of characteristics comprising a second value of the first characteristic and a second value of the second characteristic. In some embodiments, a system comprises a third luminescent label having a third ordered pair of characteristics comprising a third value of the first characteristic and a third value of the second characteristic. In certain embodiments, the first ordered pair, the second ordered pair, and the third ordered pair differ from one another in at least one of the respective values of the first and/or second characteristics. In certain embodiments, the first ordered pair, the second ordered pair, and the third ordered pair are separated by a certain minimum distance.

In some embodiments, a method comprises providing a first luminescent label having a first ordered pair of characteristics comprising a first value of a first characteristic and a first value of a second characteristic. In some embodiments, the method comprises providing a second luminescent label having a second ordered pair of characteristics comprising a second value of the first characteristic and a second value of the second characteristic. In some embodiments, the method comprises providing a third luminescent label comprising a luminescently labeled oligonucleotide structure comprising a first single-stranded oligonucleotide comprising one or more first fluorophores and a first complementary single-stranded oligonucleotide comprising one or more second fluorophores, wherein the third luminescent label has a third ordered pair of characteristics comprising a third value of the first characteristic and a third value of the second characteristic. In some embodiments, the method comprises modifying the numbers and/or identities of the one or more first fluorophores and/or the one or more second fluorophores such that the first ordered pair, the second ordered pair, and the third ordered pair differ from one another in at least one of the respective values of the first and/or second characteristics.

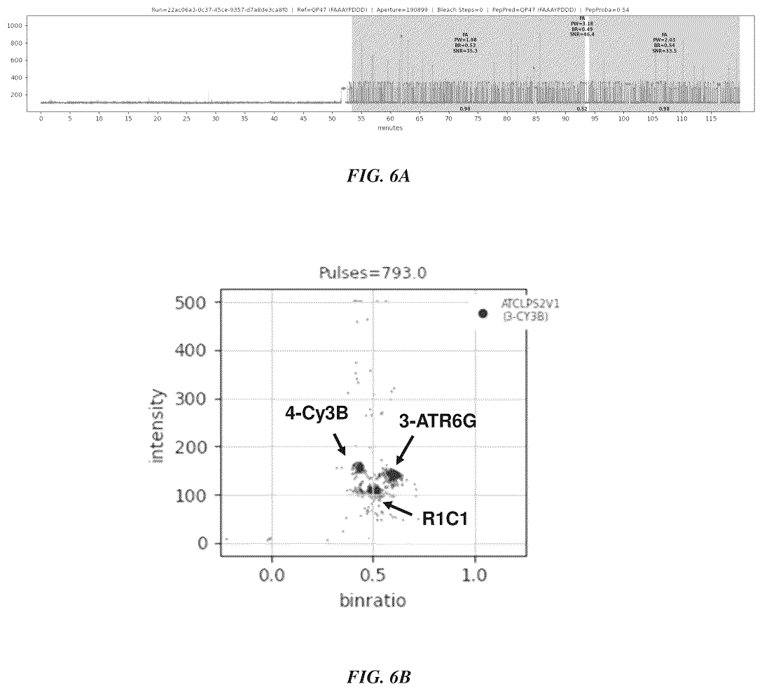

In some instances, a set of luminescent labels comprises a plurality of luminescent labels, where each luminescent label occupies of a distinct spatial region (e.g., a different location) of a two-dimensional plot of two luminescence characteristics. In certain instances, the two-dimensional plot is a plot of intensity vs. bin ratio. Non-limiting examples of plots of intensity vs. bin ratio are shown in FIGS. 6B, 7B, and 8B. In some embodiments, an ordered pair of characteristics associated with a luminescent label represents a centroid of a cluster of points associated with the luminescent label on a two-dimensional plot of two luminescence characteristics.

In some embodiments, a set of luminescent labels comprises one or more, two or more, three or more, four or more, or five or more of a first luminescent label comprising R1C1, a second luminescent label comprising C2C, a third luminescent label comprising SG4Cy3, a fourth luminescent label comprising one or more copies of ATRho6G, and a fifth luminescent label comprising one or more copies of Cy3B.

### Polypeptide Sequencing

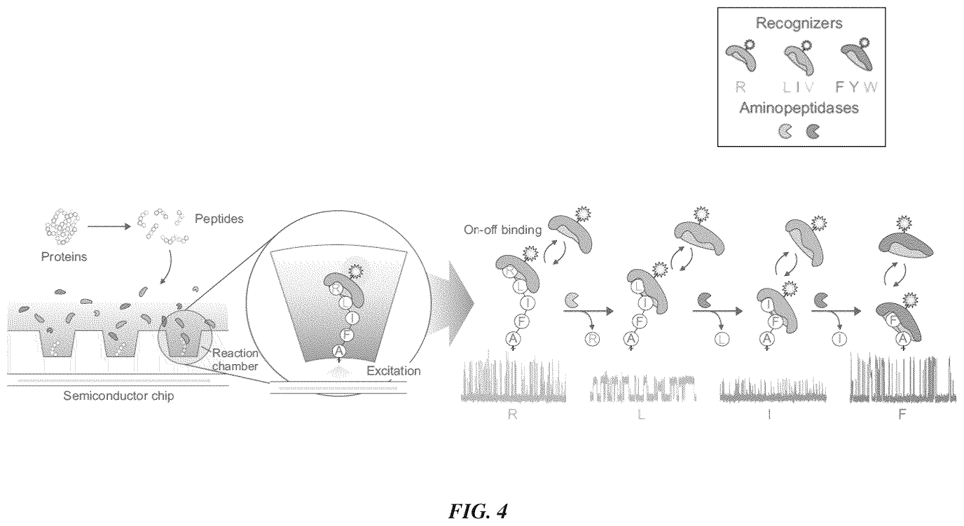

As described herein, in some aspects, the disclosure provides compositions and methods for polypeptide sequencing. FIG. 4 shows a schematic illustration of an exemplary dynamic peptide sequencing reaction in which individual on-off binding events give rise to signal pulses of a signal output. As shown at left, a polypeptide sample may be fragmented into peptides, which are immobilized in sample wells of an array, where the immobilized peptides are exposed to one or more amino acid recognition molecules (also referred to as recognizers) and one or more cleaving reagents (e.g., aminopeptidases). As shown at right, an amino acid recognition molecule reversibly binds a terminal end of the peptide, and a detectable signal is produced while the recognition molecule is bound to the peptide. As the on-off binding of recognition molecules generally occurs at a faster rate than amino acid cleavage, the binding events preceding amino acid cleavage give rise to a series of signal pulses that can be used to determine at least one chemical characteristic of the peptide (and/or an originating polypeptide). In certain embodiments, determining at least one chemical characteristic of the peptide comprises detecting the presence or absence of a target residue. In certain embodiments, determining at least one chemical characteristic of the peptide comprises determining the location of a target residue in the peptide (and/or an originating polypeptide). In certain embodiments, determining at least one chemical characteristic of the peptide comprises determining if one or more amino acids comprise a post-translational modification. In certain embodiments, determining at least one chemical characteristic of the peptide comprises determining an identity of one or more amino acids of the peptide.

Methods, reagents, and compositions for performing dynamic sequencing are described more fully in PCT International Application No. PCT/US2019/061831, filed Nov. 15, 2019, PCT International Application No. PCT/US2021/033493, filed May 20, 2021, a U.S. application entitled “Polypeptidyl Linkers,” filed on even date herewith, and a U.S. application entitled “Polypeptide Cleaving Reagents and Uses Thereof,” filed on even date herewith, each of which is incorporated herein by reference in its entirety.

Accordingly, in some embodiments, polypeptide sequencing is performed by detecting a series of signal pulses indicative of association of one or more amino acid recognition molecules with successive amino acids exposed at the terminus of a polypeptide in an ongoing degradation reaction. The series of signal pulses can be analyzed to determine characteristic patterns in the series of signal pulses, and the time course of characteristic patterns can be used to determine an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognition molecule to cleaving reagent, ratio of one recognition molecule to another, ratio of one cleaving reagent to another), number of different reagent types (e.g., the number of different types of recognition molecules and/or cleaving reagents, the number of recognition molecule types relative to the number of cleaving reagent types), cleavage activity (e.g., peptidase activity), binding properties (e.g., kinetic and/or thermodynamic binding parameters for recognition molecule binding), reagent modification (e.g., polyol and other protein modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the disclosure is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. In some embodiments, a polypeptide sequencing reaction is performed in a reaction mixture at a pH of between about 6.5 and about 9.0. In some embodiments, a polypeptide sequencing reaction is performed in a reaction mixture at a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

In some embodiments, polypeptide sequencing in accordance with the disclosure may be carried out by contacting a polypeptide with a sequencing reaction mixture comprising one or more amino acid recognition molecules and/or one or more cleaving reagents (e.g., peptidases). In some embodiments, a sequencing reaction mixture comprises an amino acid recognition molecule at a concentration of between about 10 nM and about 10 μM. In some embodiments, a sequencing reaction mixture comprises a cleaving reagent at a concentration of between about 500 nM and about 500 μM.

In some embodiments, a sequencing reaction mixture comprises an amino acid recognition molecule at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a sequencing reaction mixture comprises an amino acid recognition molecule at a concentration of about 100 nM, about 250 nM, about 500 nM, about 750 nM, or about 1 μM.

In some embodiments, a sequencing reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a sequencing reaction mixture comprises a cleaving reagent at a concentration of about 1 μM, about 5 μM, about 10 μM, about 30 μM, about 50 μM, about 70 μM, or about 100 μM.

In some embodiments, a sequencing reaction mixture comprises an amino acid recognition molecule at a concentration of between about 10 nM and about 10 μM, and a cleaving reagent at a concentration of between about 500 nM and about 500 μM. In some embodiments, a sequencing reaction mixture comprises an amino acid recognition molecule at a concentration of between about 100 nM and about 1 μM, and a cleaving reagent at a concentration of between about 1 μM and about 100 μM. In some embodiments, a sequencing reaction mixture comprises an amino acid recognition molecule at a concentration of between about 250 nM and about 1 μM, and a cleaving reagent at a concentration of between about 10 μM and about 100 μM. In some embodiments, a sequencing reaction mixture comprises an amino acid recognition molecule at a concentration of about 500 nM, and a cleaving reagent at a concentration of between about 25 μM and about 75 μM. In some embodiments, the concentration of an amino acid recognition molecule and/or the concentration of a cleaving reagent in a reaction mixture is as described elsewhere herein.

In some embodiments, a sequencing reaction mixture comprises an amino acid recognition molecule and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a sequencing reaction mixture comprises an amino acid recognition molecule and a cleaving reagent in a molar ratio of between about 10:1 and about 200:1. In some embodiments, a sequencing reaction mixture comprises an amino acid recognition molecule and a cleaving reagent in a molar ratio of between about 50:1 and about 150:1. In some embodiments, the molar ratio of an amino acid recognition molecule to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognition molecule to a cleaving reagent in a reaction mixture is between about 1:100 and about 1:1 or between about 1:1 and about 10:1. In some embodiments, the molar ratio of an amino acid recognition molecule to a cleaving reagent in a reaction mixture is as described elsewhere herein.

In some embodiments, a sequencing reaction mixture comprises one or more amino acid recognition molecules and one or more cleaving reagents. In some embodiments, a sequencing reaction mixture comprises at least three amino acid recognition molecules and at least one cleaving reagent. In some embodiments, the sequencing reaction mixture comprises two or more cleaving reagents. In some embodiments, the sequencing reaction mixture comprises at least one and up to ten cleaving reagents (e.g., 1-3 cleaving reagents, 2-10 cleaving reagents, 1-5 cleaving reagents, 3-10 cleaving reagents). In some embodiments, the sequencing reaction mixture comprises at least three and up to thirty amino acid recognition molecules (e.g., between 3 and 25, between 3 and 20, between 3 and 10, between 3 and 5, between 5 and 30, between 5 and 20, between 5 and 10, or between 10 and 20, amino acid recognition molecules).

In some embodiments, a sequencing reaction mixture comprises more than one amino acid recognition molecule and/or more than one cleaving reagent. In some embodiments, a sequencing reaction mixture described as comprising more than one amino acid recognition molecule (or cleaving reagent) refers to the mixture as having more than one type of amino acid recognition molecule (or cleaving reagent). For example, in some embodiments, a sequencing reaction mixture comprises two or more amino acid binding proteins. In some embodiments, the two or more amino acid binding proteins refer to two or more types of amino acid binding proteins. In some embodiments, one type of amino acid binding protein has an amino acid sequence that is different from another type of amino acid binding protein in the reaction mixture. In some embodiments, one type of amino acid binding protein has a label that is different from a label of another type of amino acid binding protein in the reaction mixture. In some embodiments, one type of amino acid binding protein associates with (e.g., binds to) an amino acid that is different from an amino acid with which another type of amino acid binding protein in the reaction mixture associates. In some embodiments, one type of amino acid binding protein associates with (e.g., binds to) a subset of amino acids that is different from a subset of amino acids with which another type of amino acid binding protein in the reaction mixture associates.

### Amino Acid Recognition Molecules

In some embodiments, methods provided herein comprise contacting a polypeptide with an amino acid recognition molecule, which may or may not comprise a label, that selectively binds at least one type of terminal amino acid. As used herein, in some embodiments, a terminal amino acid may refer to an amino-terminal amino acid of a polypeptide or a carboxy-terminal amino acid of a polypeptide. In some embodiments, a labeled recognition molecule selectively binds one type of terminal amino acid over other types of terminal amino acids. In some embodiments, a labeled recognition molecule selectively binds one type of terminal amino acid over an internal amino acid of the same type. In yet other embodiments, a labeled recognition molecule selectively binds one type of amino acid at any position of a polypeptide, e.g., the same type of amino acid as a terminal amino acid and an internal amino acid.

As used herein, in some embodiments, a type of amino acid refers to one of the twenty naturally occurring amino acids or a subset of types thereof. In some embodiments, a type of amino acid refers to a modified variant of one of the twenty naturally occurring amino acids or a subset of unmodified and/or modified variants thereof. Examples of modified amino acid variants include, without limitation, post-translationally-modified variants (e.g., acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination), chemically modified variants, unnatural amino acids, and proteinogenic amino acids such as selenocysteine and pyrrolysine. In some embodiments, a subset of types of amino acids includes more than one and fewer than twenty amino acids having one or more similar biochemical properties. For example, in some embodiments, a type of amino acid refers to one type selected from amino acids with charged side chains (e.g., positively and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, methods provided herein comprise contacting a polypeptide with one or more labeled recognition molecules that selectively bind one or more types of terminal amino acids. As an illustrative and non-limiting example, where four labeled recognition molecules are used in a method of the disclosure, any one recognition molecule selectively binds one type of terminal amino acid that is different from another type of amino acid to which any of the other three selectively binds (e.g., a first recognition molecule binds a first type, a second recognition molecule binds a second type, a third recognition molecule binds a third type, and a fourth recognition molecule binds a fourth type of terminal amino acid). For the purposes of this discussion, one or more labeled recognition molecules in the context of a method described herein may be alternatively referred to as a set of labeled recognition molecules.

In some embodiments, a set of labeled recognition molecules comprises at least one and up to six labeled recognition molecules. For example, in some embodiments, a set of labeled recognition molecules comprises one, two, three, four, five, or six labeled recognition molecules. In some embodiments, a set of labeled recognition molecules comprises ten or fewer labeled recognition molecules. In some embodiments, a set of labeled recognition molecules comprises eight or fewer labeled recognition molecules. In some embodiments, a set of labeled recognition molecules comprises six or fewer labeled recognition molecules. In some embodiments, a set of labeled recognition molecules comprises four or fewer labeled recognition molecules. In some embodiments, a set of labeled recognition molecules comprises three or fewer labeled recognition molecules. In some embodiments, a set of labeled recognition molecules comprises two or fewer labeled recognition molecules. In some embodiments, a set of labeled recognition molecules comprises four labeled recognition molecules. In some embodiments, a set of labeled recognition molecules comprises at least two and up to twenty (e.g., at least two and up to ten, at least two and up to eight, at least four and up to twenty, at least four and up to ten) labeled recognition molecules. In some embodiments, a set of labeled recognition molecules comprises more than twenty (e.g., 20 to 25, 20 to 30) recognition molecules. It should be appreciated, however, that any number of recognition molecules may be used in accordance with a method of the disclosure to accommodate a desired use.

In accordance with the disclosure, in some embodiments, one or more types of amino acids are identified by detecting luminescence of a labeled recognition molecule. In some embodiments, a labeled recognition molecule comprises a recognition molecule that selectively binds one type of amino acid and a luminescent label having a luminescence that is associated with the recognition molecule. In this way, the luminescence (e.g., luminescence lifetime, luminescence intensity, and other luminescence properties described elsewhere herein) may be associated with the selective binding of the recognition molecule to identify an amino acid of a polypeptide. In some embodiments, a plurality of types of labeled recognition molecules may be used in a method according to the disclosure, where each type comprises a luminescent label having a luminescence that is uniquely identifiable from among the plurality. In some embodiments, the luminescent label of each type of labeled recognition molecule is uniquely identifiable from among the plurality by luminescence intensity alone. Suitable luminescent labels may include luminescent molecules, such as fluorophore dyes, and are described elsewhere herein.

In some embodiments, an amino acid recognition molecule may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the disclosure provides methods of sequencing by detecting reversible binding interactions during a polypeptide degradation process. Advantageously, such methods may be performed using a recognition molecule that reversibly binds with low affinity to more than one type of amino acid (e.g., a subset of amino acid types).

As used herein, in some embodiments, the terms “selective” and “specific” (and variations thereof, e.g., selectively, specifically, selectivity, specificity) refer to a preferential binding interaction. For example, in some embodiments, an amino acid recognition molecule that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). Accordingly, it should be appreciated that a selective binding interaction can refer to any binding interaction that is uniquely identifiable to one type of amino acid over other types of amino acids. For example, in some aspects, the disclosure provides methods of polypeptide sequencing by obtaining data indicative of association of one or more amino acid recognition molecules with a polypeptide molecule. In some embodiments, the data comprises a series of signal pulses corresponding to a series of reversible amino acid recognition molecule binding interactions with an amino acid of the polypeptide molecule, and the data may be used to determine the identity of the amino acid. As such, in some embodiments, a “selective” or “specific” binding interaction refers to a detected binding interaction that discriminates between one type of amino acid and other types of amino acids.

In some embodiments, an amino acid recognition molecule binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, an amino acid recognition molecule binds one type of amino acid (e.g., one type of terminal amino acid) with a K_{D} of less than about 100 nM, less than about 50 nM, less than about 25 nM, less than about 10 nM, or less than about 1 nM. In some embodiments, an amino acid recognition molecule binds one type of amino acid with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds one type of amino acid with a K_{D} of about 50 nM.

In some embodiments, an amino acid recognition molecule binds two or more types of amino acids with a K_{D} of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M). In some embodiments, an amino acid recognition molecule binds two or more types of amino acids with a K_{D} of less than about 100 nM, less than about 50 nM, less than about 25 nM, less than about 10 nM, or less than about 1 nM. In some embodiments, an amino acid recognition molecule binds two or more types of amino acids with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds two or more types of amino acids with a K_{D} of about 50 nM.

In some embodiments, an amino acid recognition molecule binds at least one type of amino acid with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or k_{off} can be a known literature value, or the value can be determined empirically. In some embodiments, the value for k_{off} can be determined empirically based on signal pulse information obtained in a single-molecule assay as described elsewhere herein. For example, the value for k_{off} can be approximated by the reciprocal of the mean pulse duration. In some embodiments, an amino acid recognition molecule binds two or more types of amino acids with a different K_{D} or k_{off} for each of the two or more types. In some embodiments, a first K_{D} or k_{off} for a first type of amino acid differs from a second K_{D} or k_{off} for a second type of amino acid by at least 10% (e.g., at least 25%, at least 50%, at least 100%, or more). In some embodiments, the first and second values for K_{D} or k_{off} differ by about 10-25%, 25-50%, 50-75%, 75-100%, or more than 100%, for example by about 2-fold, 3-fold, 4-fold, 5-fold, or more.

As described herein, an amino acid recognition molecule may be any biomolecule capable of selectively or specifically binding one molecule over another molecule (e.g., one type of amino acid over another type of amino acid). In some embodiments, a recognition molecule is not a peptidase or does not have peptidase activity. For example, in some embodiments, methods of polypeptide sequencing of the disclosure involve contacting a polypeptide molecule with one or more recognition molecules and a cleaving reagent. In such embodiments, the one or more recognition molecules do not have peptidase activity, and removal of one or more amino acids from the polypeptide molecule (e.g., amino acid removal from a terminus of the polypeptide molecule) is performed by the cleaving reagent.

Recognition molecules include, for example, proteins and nucleic acids, which may be synthetic or recombinant. In some embodiments, a recognition molecule may be an antibody or an antigen-binding portion of an antibody, an SH2 domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a recognition molecule of the disclosure is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule is an N-end rule pathway protein selected from a Gid protein (e.g., Gid4 or Gid10 protein), a UBR-box protein (e.g., UBR1, UBR2) or UBR-box domain-containing protein fragment thereof, a p62 protein or ZZ domain-containing fragment thereof, and a ClpS protein (e.g., ClpS1, ClpS2). Accordingly, in some embodiments, a labeled recognition molecule comprises a degradation pathway protein. In some embodiments, a labeled recognition molecule comprises a ClpS protein.

In some embodiments, a recognition molecule of the disclosure is a ClpS protein, such as Agrobacterium tumifaciens ClpS 1, Agrobacterium tumifaciens ClpS2, Synechococcus elongatus ClpS 1, Synechococcus elongatus ClpS2, Thermosynechococcus elongatus ClpS, Escherichia coli ClpS, or Plasmodium falciparum ClpS. In some embodiments, the recognition molecule is an L/F transferase, such as Escherichia coli leucyl/phenylalanyl-tRNA-protein transferase. In some embodiments, the recognition molecule is a D/E leucyltransferase, such as Vibrio vulnificus Aspartate/glutamate leucyltransferase Bpt. In some embodiments, the recognition molecule is a UBR protein or UBR-box domain, such as the UBR protein or UBR-box domain of human UBR1 and UBR2 or Saccharomyces cerevisiae UBR1. In some embodiments, the recognition molecule is a p62 protein, such as H. sapiens p62 protein or Rattus norvegicus p62 protein, or truncation variants thereof that minimally include a ZZ domain. In some embodiments, the recognition molecule is a Gid4 protein, such as H. sapiens GID4 or Saccharomyces cerevisiae GID4. In some embodiments, the recognition molecule is a Gid10 protein, such as Saccharomyces cerevisiae GID10. In some embodiments, the recognition molecule is an N-meristoyltransferase, such as Leishmania major N-meristoyltransferase or H. sapiens N-meristoyltransferase NMT1. In some embodiments, the recognition molecule is a BIR2 protein, such as Drosophila melanogaster BIR2. In some embodiments, the recognition molecule is a tyrosine kinase or SH2 domain of a tyrosine kinase, such as H. sapiens Fyn SH2 domain, H. sapiens Src tyrosine kinase SH2 domain, or variants thereof, such as H. sapiens Fyn SH2 domain triple mutant superbinder. In some embodiments, the recognition molecule is an antibody or antibody fragment, such as a single-chain antibody variable fragment (scFv) against phosphotyrosine or another post-translationally modified amino acid variant described herein.

In some embodiments, a recognition molecule of the disclosure is an amino acid binding protein which can be used with other types of amino acid binding molecules, such as a peptidase and/or a nucleic acid aptamer, in a method sequencing. A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a labeled recognition molecule comprises a peptidase that has been modified to inactivate exopeptidase or endopeptidase activity. In this way, the labeled recognition molecule selectively binds without also cleaving the amino acid from a polypeptide. In yet other embodiments, a peptidase that has not been modified to inactivate exopeptidase or endopeptidase activity may be used with an amino acid binding protein of the disclosure. For example, in some embodiments, a labeled recognition molecule comprises a labeled exopeptidase.

In some embodiments, an amino acid recognition molecule comprises one or more labels. In some embodiments, the one or more labels comprise a luminescent label or a conductivity label as described elsewhere herein. In some embodiments, the one or more labels comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognition molecule is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognition molecule with other recognition molecules, with a cleaving reagent, or with other species present in a sequencing reaction mixture. PEGylation can be performed by incubating a recognition molecule (e.g., an amino acid binding protein, such as a ClpS protein) with mPEG4-NHS ester, which labels primary amines such as surface-exposed lysine side chains. Other types of PEG and other methods of polyol modification are known in the art.

In some embodiments, the one or more labels comprise a tag sequence. For example, in some embodiments, an amino acid recognition molecule comprises a tag sequence that provides one or more functions other than amino acid binding. In some embodiments, a tag sequence comprises at least one biotin ligase recognition sequence that permits biotinylation of the recognition molecule (e.g., incorporation of one or more biotin molecules, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem.

Additional examples of functional sequences in a tag sequence include purification tags, cleavage sites, and other moieties useful for purification and/or modification of recognition molecules.

Examples of amino acid recognition molecules (e.g., amino acid binding proteins) for use in accordance with the disclosure are described more fully in PCT International Application No. PCT/US2019/061831, filed Nov. 15, 2019, and PCT International Application No. PCT/US2021/033493, filed May 20, 2021, the relevant content of which is incorporated herein by reference in its entirety.

### Cleaving Reagents

In some embodiments, a cleaving reagent of the disclosure is an exopeptidase. An exopeptidase generally requires a polypeptide substrate to comprise at least one of a free amino group at its amino-terminus or a free carboxyl group at its carboxy-terminus. In some embodiments, an exopeptidase in accordance with the disclosure hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the disclosure is an aminopeptidase or a carboxypeptidase, which cleaves a single amino acid from an amino- or a carboxy-terminus, respectively. In some embodiments, an exopeptidase in accordance with the disclosure is a dipeptidyl-peptidase or a peptidyl-dipeptidase, which cleave a dipeptide from an amino- or a carboxy-terminus, respectively. In yet other embodiments, an exopeptidase in accordance with the disclosure is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stöcker, W. Proteases: Structure and Function. Chapter 1). In some embodiments, a peptidase in accordance with the disclosure removes more than three amino acids from a polypeptide terminus. Accordingly, in some embodiments, the peptidase is an endopeptidase, e.g., that cleaves preferentially at particular positions (e.g., before or after a particular amino acid). In some embodiments, the size of a polypeptide cleavage product of endopeptidase activity will depend on the distribution of cleavage sites (e.g., amino acids) within the polypeptide being analyzed.

An exopeptidase in accordance with the disclosure may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids, which may be used as labeled exopeptidases or inactivated to be used as non-cleaving labeled recognition molecules described herein, have been described in the literature (see, e.g., Garcia-Guerrero, M.C., et al. (2018) PNAS 115(17)).

Suitable peptidases for use as cleaving reagents and/or recognition molecules include aminopeptidases that selectively bind one or more types of amino acids. In some embodiments, an aminopeptidase recognition molecule is modified to inactivate aminopeptidase activity. In some embodiments, an aminopeptidase cleaving reagent is non-specific such that it cleaves most or all types of amino acids from a terminal end of a polypeptide. In some embodiments, an aminopeptidase cleaving reagent is more efficient at cleaving one or more types of amino acids from a terminal end of a polypeptide as compared to other types of amino acids at the terminal end of the polypeptide. For example, an aminopeptidase in accordance with the disclosure specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase.

In some embodiments, an aminopeptidase is a non-specific aminopeptidase. In some embodiments, a non-specific aminopeptidase is a zinc metalloprotease.

Examples of cleaving reagents (e.g., aminopeptidases) for use in accordance with the disclosure are described more fully in PCT International Application No. PCT/US2019/061831, filed Nov. 15, 2019, and PCT International Application No. PCT/US2021/033493, filed May 20, 2021, the relevant content of which is incorporated herein by reference in its entirety.

### Nucleic Acid Sequencing

Some aspects of the application are useful for sequencing biological polymers, such as nucleic acids. In some embodiments, methods, compositions, and devices described in the application can be used to identify a series of nucleotide monomers that are incorporated into a nucleic acid (e.g., by detecting a time-course of incorporation of a series of labeled nucleotide). In some embodiments, methods, compositions, and devices described in the application can be used to identify a series of nucleotides that are incorporated into a template-dependent nucleic acid sequencing reaction product synthesized by a polymerase enzyme.

In certain embodiments, the template-dependent nucleic acid sequencing product is carried out by naturally occurring nucleic acid polymerases. In some embodiments, the polymerase is a mutant or modified variant of a naturally occurring polymerase. In some embodiments, the template-dependent nucleic acid sequence product will comprise one or more nucleotide segments complementary to the template nucleic acid strand. In one aspect, the application provides a method of determining the sequence of a template (or target) nucleic acid strand by determining the sequence of its complementary nucleic acid strand.

In another aspect, the application provides methods of sequencing target nucleic acids by sequencing a plurality of nucleic acid fragments, wherein the target nucleic acid comprises the fragments. In certain embodiments, the method comprises combining a plurality of fragment sequences to provide a sequence or partial sequence for the parent target nucleic acid. In some embodiments, the step of combining is performed by computer hardware and software. The methods described herein may allow for a set of related target nucleic acids, such as an entire chromosome or genome to be sequenced.

During sequencing, a polymerizing enzyme may couple (e.g., attach) to a priming location of a target nucleic acid molecule. The priming location can be a primer that is complementary to a portion of the target nucleic acid molecule. As an alternative the priming location is a gap or nick that is provided within a double stranded segment of the target nucleic acid molecule. A gap or nick can be from 0 to at least 1, 2, 3, 4, 5, 6, 7, 8, 9, 10, 20, 30, or 40 nucleotides in length. A nick can provide a break in one strand of a double stranded sequence, which can provide a priming location for a polymerizing enzyme, such as, for example, a strand displacing polymerase enzyme.

In some cases, a sequencing primer can be annealed to a target nucleic acid molecule that may or may not be immobilized to a solid support. A solid support can comprise, for example, a sample well (e.g., a nanoaperture, a reaction chamber) on a chip used for nucleic acid sequencing. In some embodiments, a sequencing primer may be immobilized to a solid support and hybridization of the target nucleic acid molecule also immobilizes the target nucleic acid molecule to the solid support. In some embodiments, a polymerase is immobilized to a solid support and soluble primer and target nucleic acid are contacted to the polymerase. However, in some embodiments a complex comprising a polymerase, a target nucleic acid and a primer is formed in solution and the complex is immobilized to a solid support (e.g., via immobilization of the polymerase, primer, and/or target nucleic acid). In some embodiments, none of the components in a sample well (e.g., a nanoaperture, a reaction chamber) are immobilized to a solid support. For example, in some embodiments, a complex comprising a polymerase, a target nucleic acid, and a primer is formed in solution and the complex is not immobilized to a solid support.

Under appropriate conditions, a polymerase enzyme that is contacted to an annealed primer/target nucleic acid can add or incorporate one or more nucleotides onto the primer, and nucleotides can be added to the primer in a 5′ to 3′, template-dependent fashion. Such incorporation of nucleotides onto a primer (e.g., via the action of a polymerase) can generally be referred to as a primer extension reaction. Each nucleotide can be associated with a detectable tag that can be detected and identified (e.g., based on its luminescent lifetime and/or other characteristics) during the nucleic acid extension reaction and used to determine each nucleotide incorporated into the extended primer and, thus, a sequence of the newly synthesized nucleic acid molecule. Via sequence complementarity of the newly synthesized nucleic acid molecule, the sequence of the target nucleic acid molecule can also be determined. In some cases, annealing of a sequencing primer to a target nucleic acid molecule and incorporation of nucleotides to the sequencing primer can occur at similar reaction conditions (e.g., the same or similar reaction temperature) or at differing reaction conditions (e.g., different reaction temperatures). In some embodiments, sequencing by synthesis methods can include the presence of a population of target nucleic acid molecules (e.g., copies of a target nucleic acid) and/or a step of amplification of the target nucleic acid to achieve a population of target nucleic acids. However, in some embodiments sequencing by synthesis is used to determine the sequence of a single molecule in each reaction that is being evaluated (and nucleic acid amplification is not required to prepare the target template for sequencing). In some embodiments, a plurality of single molecule sequencing reactions are performed in parallel (e.g., on a single chip) according to aspects of the present application. For example, in some embodiments, a plurality of single molecule sequencing reactions are each performed in separate reaction chambers (e.g., nanoapertures, sample wells) on a single chip.

Embodiments are capable of sequencing single nucleic acid molecules with high accuracy and long read lengths, such as an accuracy of at least about 50%, 60%, 70%, 75%, 80%, 85%, 90%, 95%, 96%, 97%, 98%, 99%, 99.9%, 99.99%, 99.999%, or 99.9999%, and/or read lengths greater than or equal to about 10 base pairs (bp), 50 bp, 100 bp, 200 bp, 300 bp, 400 bp, 500 bp, 1000 bp, 10,000 bp, 20,000 bp, 30,000 bp, 40,000 bp, 50,000 bp, or 100,000 bp. In some embodiments, the target nucleic acid molecule used in single molecule sequencing is a single stranded target nucleic acid (e.g., deoxyribonucleic acid (DNA), DNA derivatives, ribonucleic acid (RNA), RNA derivatives) template that is added or immobilized to a sample well (e.g., nanoaperture) containing at least one additional component of a sequencing reaction (e.g., a polymerase such as, a DNA polymerase, a sequencing primer) immobilized or attached to a solid support such as the bottom or side walls of the sample well. The target nucleic acid molecule or the polymerase can be attached to a sample wall, such as at the bottom or side walls of the sample well directly or through a linker. The sample well (e.g., nanoaperture) also can contain any other reagents needed for nucleic acid synthesis via a primer extension reaction, such as, for example suitable buffers, co-factors, enzymes (e.g., a polymerase) and deoxyribonucleoside polyphosphates, such as, e.g., deoxyribonucleoside triphosphates, including deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include luminescent tags, such as fluorophores. In some embodiments, each class of dNTPs (e.g., adenine-containing dNTPs (e.g., dATP), cytosine-containing dNTPs (e.g., dCTP), guanine-containing dNTPs (e.g., dGTP), uracil-containing dNTPs (e.g., dUTPs) and thymine-containing dNTPs (e.g., dTTP)) is conjugated to a distinct luminescent tag such that detection of light emitted from the tag indicates the identity of the dNTP that was incorporated into the newly synthesized nucleic acid. Emitted light from the luminescent tag can be detected and attributed to its appropriate luminescent tag (and, thus, associated dNTP) via any suitable device and/or method, including such devices and methods for detection described elsewhere herein. The luminescent tag may be conjugated to the dNTP at any position such that the presence of the luminescent tag does not inhibit the incorporation of the dNTP into the newly synthesized nucleic acid strand or the activity of the polymerase. In some embodiments, the luminescent tag is conjugated to the terminal phosphate (e.g., the gamma phosphate) of the dNTP.

In some embodiments, the single-stranded target nucleic acid template can be contacted with a sequencing primer, dNTPs, polymerase and other reagents necessary for nucleic acid synthesis. In some embodiments, all appropriate dNTPs can be contacted with the single-stranded target nucleic acid template simultaneously (e.g., all dNTPs are simultaneously present) such that incorporation of dNTPs can occur continuously. In other embodiments, the dNTPs can be contacted with the single-stranded target nucleic acid template sequentially, where the single-stranded target nucleic acid template is contacted with each appropriate dNTP separately, with washing steps in between contact of the single-stranded target nucleic acid template with differing dNTPs. Such a cycle of contacting the single-stranded target nucleic acid template with each dNTP separately followed by washing can be repeated for each successive base position of the single-stranded target nucleic acid template to be identified.

In some embodiments, the sequencing primer anneals to the single-stranded target nucleic acid template and the polymerase consecutively incorporates the dNTPs (or other deoxyribonucleoside polyphosphate) to the primer based on the single-stranded target nucleic acid template. The unique luminescent tag associated with each incorporated dNTP can be excited with the appropriate excitation light during or after incorporation of the dNTP to the primer and its emission can be subsequently detected, using, any suitable device(s) and/or method(s), including devices and methods for detection described elsewhere herein. Detection of a particular emission of light (e.g., having a particular emission lifetime, intensity, spectrum and/or combination thereof) can be attributed to a particular dNTP incorporated. The sequence obtained from the collection of detected luminescent tags can then be used to determine the sequence of the single-stranded target nucleic acid template via sequence complementarity.

While the present disclosure makes reference to dNTPs, devices, systems and methods provided herein may be used with various types of nucleotides, such as ribonucleotides and deoxyribonucleotides (e.g., deoxyribonucleoside polyphosphates with at least 4, 5, 6, 7, 8, 9, or 10 phosphate groups). Such ribonucleotides and deoxyribonucleotides can include various types of tags (or markers) and linkers.

### Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample well may have a suitable size and shape such that at least a portion of the sample well receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light sources external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample well, which may range in number between approximately 10,000 pixels to 1,000,000 pixels according to some embodiments, multiple samples can be analyzed in parallel.

The integrated device may include an optical system for receiving excitation light and directing the excitation light among the reaction chamber array. The optical system may include one or more grating couplers configured to couple excitation light to other optical components of the integrated device and direct the excitation light to the other optical components. For example, the optical system may include optical components that direct the excitation light from the grating coupler(s) towards the reaction chamber array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a reaction chamber and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding reaction chamber. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a reaction chamber and its respective photodetector(s) may be aligned along a common axis. In this manner, the photodetector(s) may overlap with the reaction chamber within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the reaction chambers are carried out by exciting some or all of the samples within the chambers using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each reaction chamber to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

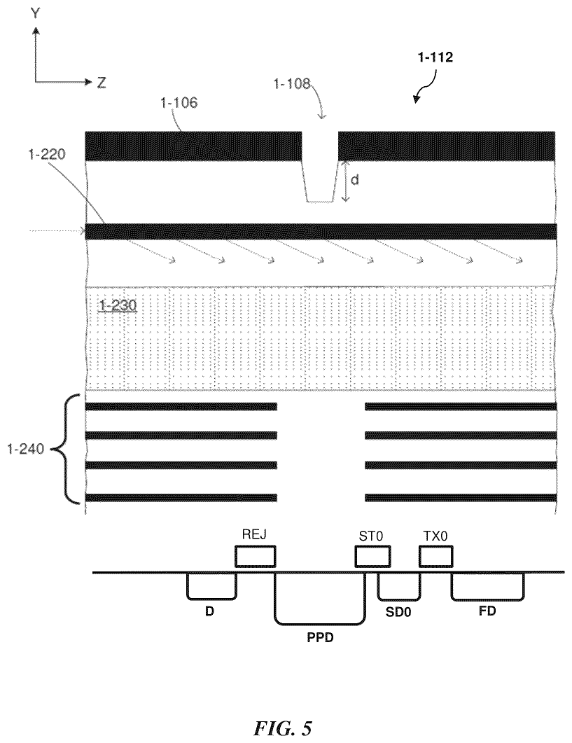

FIG. 5 illustrates a cross-sectional view of a pixel 1-112 of an integrated device 1-102. Pixel 1-112 includes a photodetection region, which may be a pinned photodiode (PPD), and a charge storage region, which may be a storage diode (SD0). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of pixel 1-112, excitation light may illuminate reaction chamber 1-108 causing incident photons, including fluorescence emissions from a sample, to flow along the optical axis to photodetection region PPD. As shown in FIG. 5, pixel 1-112 may include a waveguide 1-220 configured to optically (e.g., evanescently) couple excitation light from a grating coupler of the integrated device (not shown) to the reaction chamber 1-108. In response, a sample in the reaction chamber 1-108 may emit fluorescent light toward photodetection region PPD. In some embodiments, pixel 1-112 may also include one or more photonic structures 1-230, which may include one or more optical rejection structures such as a spectral filter, a polarization filter, and/or a spatial filter. For example, the photonic structures 1-230 may be configured to reduce the amount of excitation light that reaches the photodetection region PPD and/or increase the amount of fluorescent emissions that reach the photodetection region PPD. Also shown in pixel 1-112, pixel 1-112 may include one or more metal layers 1-240, which may be configured as a filter and/or may carry control signals from a control circuit configured to control transfer gates, as described further herein.

In some embodiments, pixel 1-112 may include one or more transfer gates configured to control operation of pixel 1-112 by applying an electrical bias to one or more semiconductor regions of pixel 1-112 in response to one or more control signals. For example, when transfer gate ST0 induces a first electrical bias at the semiconductor region between photodetection region PPD and storage region SD0, a transfer path (e.g., charge transfer channel) may be formed in the semiconductor region. Charge carriers (e.g., photo-electrons) generated in photodetection region PPD by the incident photons may flow along the transfer path to storage region SD0. In some embodiments, the first electrical bias may be applied during a collection period during which charge carriers from the sample are selectively directed to storage region SD0. Alternatively, when transfer gate ST0 provides a second electrical bias at the semiconductor region between photodetection region PPD and storage region SD0, charge carriers from photodetection region PPD may be blocked from reaching storage region SD0 along the transfer path. In some embodiments, drain gate REJ may provide a channel to drain D to draw noise charge carriers generated in photodetection region PPD by the excitation light away from photodetection region PPD and storage region SD0, such as during a rejection period before fluorescent emission photons from the sample reach photodetection region PPD. In some embodiments, during a readout period, transfer gate ST0 may provide the second electrical bias and transfer gate TX0 may provide an electrical bias to cause charge carriers stored in storage region SD0 to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the reaction chamber. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### EXAMPLES

### Example 1

A polypeptide sequencing run was performed using amino acid recognition molecules labeled with a first luminescent label comprising 4 copies of Cy®3B, a second luminescent label comprising 3 copies of ATRho6G, and a third luminescent label comprising a luminescently labeled oligonucleotide structure comprising a first single-stranded oligonucleotide comprising one copy of Cy®3B and having 100% sequence identity to Sequence A and a first complementary single-stranded oligonucleotide comprising one copy of ATRho6G and having 100% sequence identity to Sequence B (referred to as R1C1). In R1C1, the ATRho6G and Cy®3B fluorophores were separated by a distance of 10 nm. The distance was predicted from a B-DNA model and can be approximated as 0.34*n, where n is the number of oligonucleotide bases between the fluorophores.

The polypeptide sequencing run was performed for a sample peptide having the sequence FAAAYPDDD (SEQ ID NO: 17). FIG. 6A shows a representative trace demonstrating that phenylalanine (F) was identified. FIG. 6B shows a plot of intensity vs. bin ratio. From FIG. 6B, it can be seen that each of Cy®3B, ATRho6G, and R1C1 occupied distinct spatial regions of the plot. Further, FIG. 6B demonstrates that R1C1 had a bin ratio of 0.51, which fell between the 0.43 bin ratio of Cy®3B and the 0.58 bin ratio of ATRho6G.

The fact that the R1C1 bin ratio matched the average bin ratios of Cy®3B and ATRho6G demonstrated that the 10 nm distance between the ATRho6G and Cy®3B fluorophores effectively prevented FRET formation between the two fluorophores. In addition, the R1C1 bin ratio demonstrated that the contribution of the apparent fluorescence lifetime from each fluorophore was proportional to its intensity.

### Example 2

A polypeptide sequencing run was performed using amino acid recognition molecules labeled with a first luminescent label comprising 8 copies of Cy®3, a second luminescent label comprising 4 copies of Cy®3B, and a third luminescent label comprising a luminescently labeled oligonucleotide structure comprising a first single-stranded oligonucleotide comprising two copies of Cy®3 and having 100% sequence identity to Sequence C and a first complementary single-stranded oligonucleotide comprising one copy of Cy®3B and having 100% sequence identity to Sequence D (referred to as C2C). In C2C, the ATRho6G and Cy®3B fluorophores were separated by a distance of 10 nm. The distance was predicted from a B-DNA model and can be approximated as 0.34*n, where n is the number of oligonucleotide bases between the fluorophores.

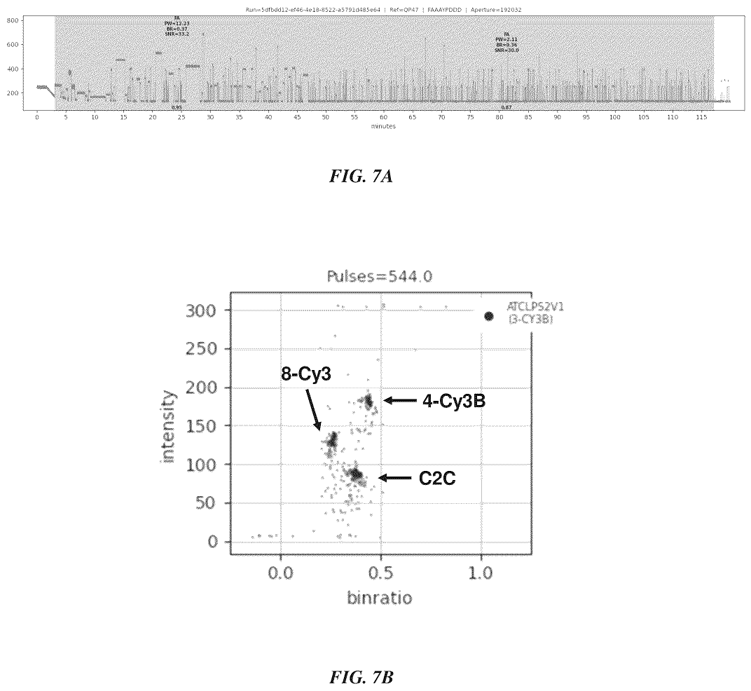

The polypeptide sequencing run was performed for a sample peptide having the sequence FAAAYPDDD (SEQ ID NO: 17). FIG. 7A shows a representative trace demonstrating that phenylalanine (F) was identified. FIG. 7B shows a plot of intensity vs. bin ratio. From FIG. 7B, it can be seen that each of Cy®3, Cy®3B, and C2C occupied distinct spatial regions of the plot. Further, FIG. 7B demonstrates that C2C had a bin ratio of 0.39, which fell between the 0.28 bin ratio of Cy®3 and the 0.44 bin ratio of Cy®3B.

The fact that the C2C bin ratio matched the average bin ratios of Cy®3 and Cy®3B demonstrated that the 10 nm distance between the Cy®3 and Cy®3B fluorophores effectively prevented FRET formation between the two fluorophores. In addition, the C2C bin ratio demonstrated that the contribution of the apparent fluorescence lifetime from each fluorophore was proportional to its intensity.

### Example 3

A polypeptide sequencing run was performed using amino acid recognition molecules labeled with a first luminescent label comprising 4 copies of Cy®3B, a second luminescent label comprising C2C, and a third luminescent label comprising a luminescently labeled oligonucleotide structure comprising a first single-stranded oligonucleotide comprising two copies of Cy®3 and having 100% sequence identity to Sequence E and a first complementary single-stranded oligonucleotide comprising two copies of Cy®3 and having 100% sequence identity to Sequence F (referred to as SG4Cy®3). In SG4Cy®3, each oligonucleotide strand had 2 Cy®3 fluorophores, which were bulged out around a GC rich region.

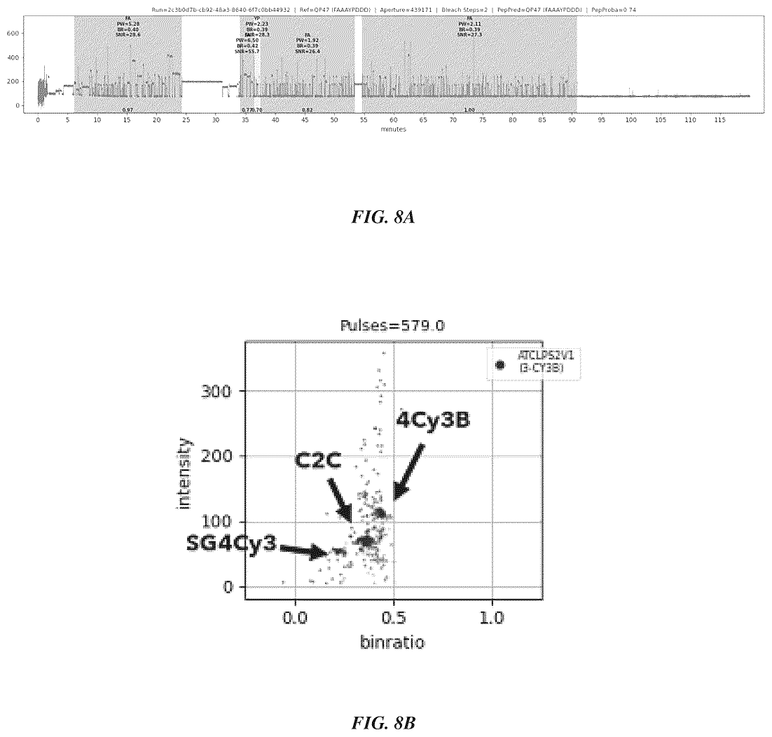

The polypeptide sequencing run was performed for a sample peptide having the sequence FAAAYPDDD (SEQ ID NO: 17). FIG. 8A shows a representative trace demonstrating that phenylalanine (F) and tyrosine (Y) residues were identified. FIG. 8B shows a plot of intensity vs. bin ratio. From FIG. 8B, it can be seen that each of Cy®3B, C2C, and SG4Cy®3 occupied distinct spatial regions of the plot.

### Example 4

Luminescently labeled oligonucleotide structures comprising multiple luminescently labeled oligonucleotides comprising multiple luminescent labels were assembled by a stepwise hybridization and conjugation approach, as schematically illustrated in FIG. 3A.

To avoid oligonucleotide duplex bending and curving and to facilitate conjugation and hybridization of a plurality of luminescently labeled oligonucleotides, two different types of oligonucleotides were used. The first type of oligonucleotide included four types of nucleotides (A, C, G, T). The first type of oligonucleotide was a “GCAT system oligonucleotide.” The second type of oligonucleotide included up to seven types of nucleotides (A, C, G, T, iG, iC, diaminopurine). The second type of oligonucleotide was a “GCATiGiC system oligonucleotide.”

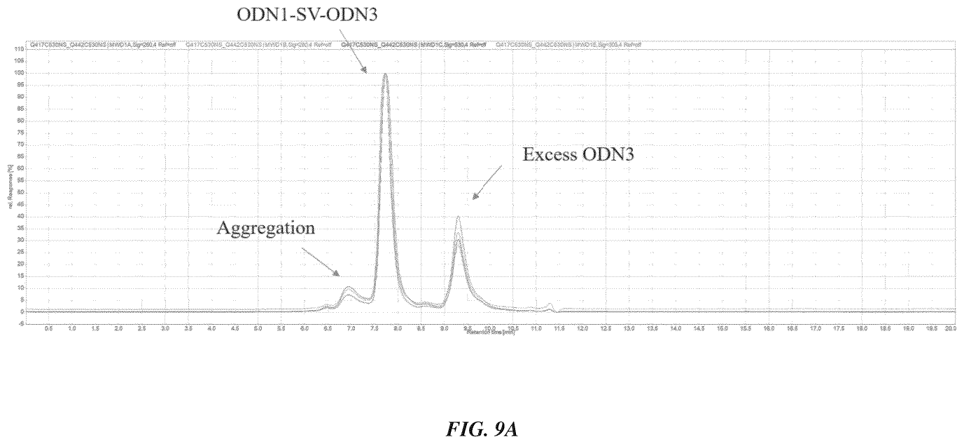

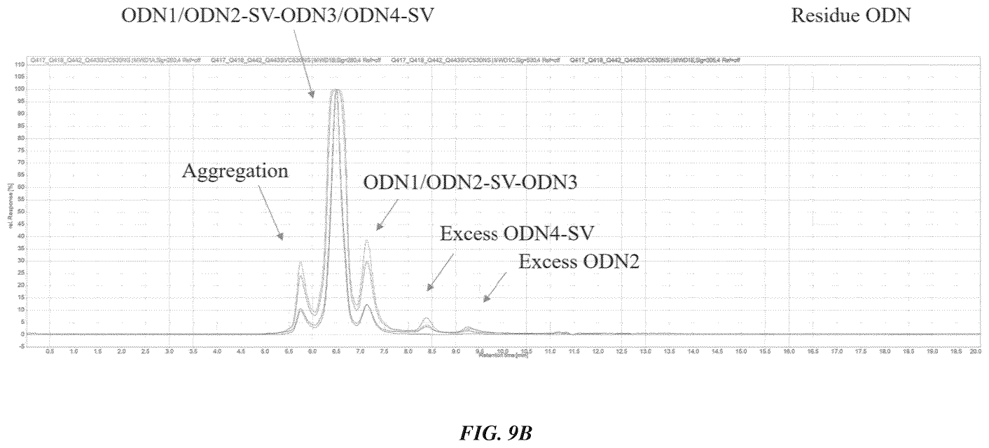

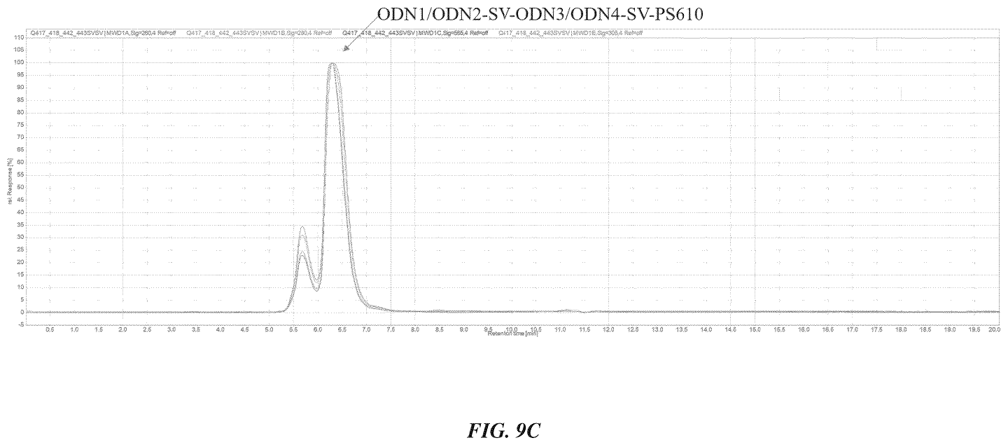

Luminescently labeled oligonucleotide structures were assembled by biotinylating a first GCAT system oligonucleotide (ODN1) and conjugating ODN1 to a one end of a streptavidin (SV) homotetramer. Next, a first GCATiGiC system oligonucleotide (ODN3) was biotinylated and conjugated to the second end of the streptavidin homotetramer forming an ODN1-SV-ODN3 intermediate structure. Both ODN1 and ODN3 were luminescently labeled. FIG. 9A shows a retention plot illustrating the ODN1-SV-ODN3 intermediate structure, as well as excess ODN3 that did not conjugate to the streptavidin. Next, a GCAT system oligonucleotide with complementarity to ODN1 (ODN2) was hybridized to ODN1 and a GCATiGiC system oligonucleotide with complementarity to ODN3 (ODN4) was hybridized to ODN3. Both ODN2 and ODN4 were luminescently labeled, and ODN4 was further conjugated to a second streptavidin. This step resulted in an ODN1/ODN2-SV-ODN3/ODN4-SV intermediate structure. FIG. 9B shows a retention plot illustrating the ODN1/ODN2-SV-ODN3/DON4-SV intermediate structure, as well as excess species that did not conjugate to the streptavidin or hybridize to the conjugated oligonucleotides. Finally, to prepare the luminescently labeled oligonucleotide structure for sequencing, a terminator was added to one end of the structure, and a biotinylated amino acid recognizer protein was added to the second streptavidin. The final step resulted in an ODN1/ODN2-SV-ODN3/ODN4-SV-PS610 structure. FIG. 9C shows a retention plot illustrating the ODN1/ODN2-SV-ODN3/ODN4-SV-PS610 structure. Sequences for ODN1, ODN2, ODN3, and ODN4 are provided in Table 2, where /X/ is C530NS.

TABLE 2

| Oligonucleotide | Name | Sequence |
|---|---|---|
| ODN1 | G | GCCATT/X/ATACGGATT/X/ |
| | | ATTCGGTTATATTGCCTATT |
| | | ATTGCG (SEQ ID NO: 8) |
| ODN2 | H | CGCAAT/X/ATAGGCAAT/X/ |
| | | TAACCGAATTAATCCGTAT |
| | | TAATGGC (SEQ ID NO: 9) |
| ODN3 | I | iGiCGTAT/X/TAAGiGGTAT/ |
| | | X/TAAGiCCAAATAATGCGTAA |
| | | TAAAGIGC (SEQ ID NO: 10) |
| ODN4 | J | GiCCTTT/X/TTACGCATT/X/ |
| | | TTTGiGCTTATATACiCCTTAT |
| | | ATACiGiC (SEQ ID NO: 11) |

### Example 5

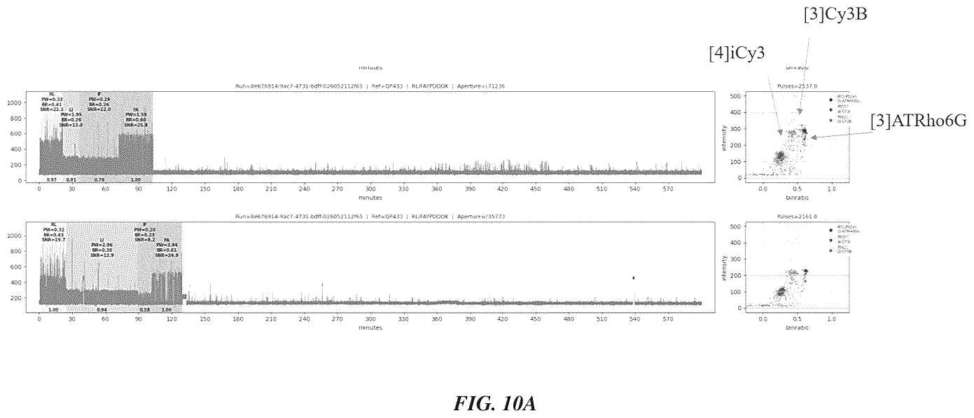

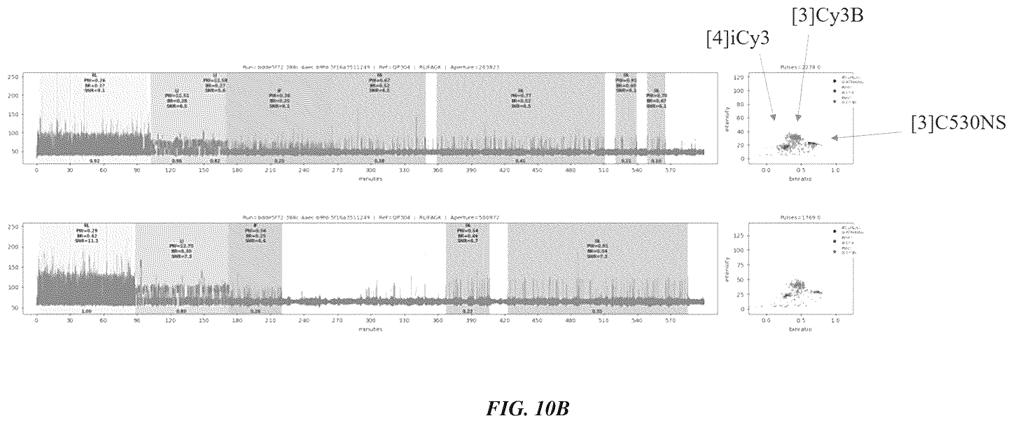

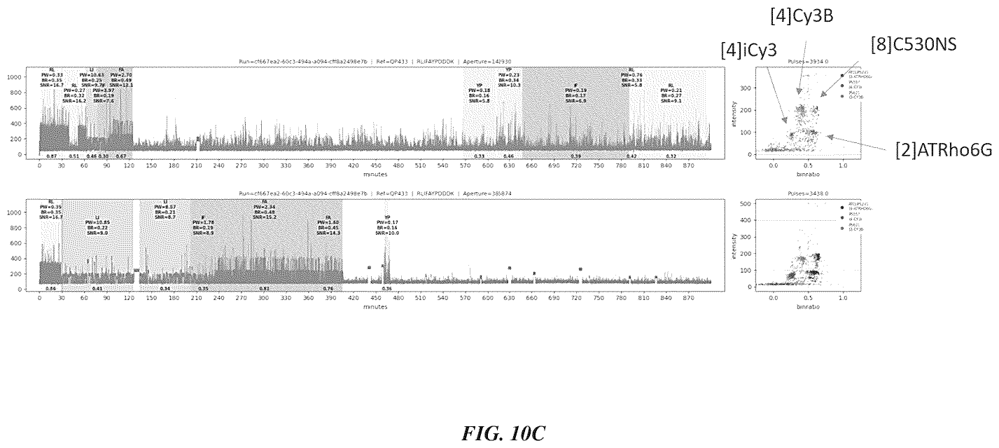

Luminescently labeled oligonucleotide structures from Example 4 were evaluated in polypeptide sequencing reactions to determine the efficacy of the structure as compared to a standard luminescently labeled oligonucleotide structure. FIG. 10A shows a representative trace from a polypeptide sequencing reaction using a first luminescent label comprising 4 copies of Cy®3, a second luminescent label comprising 3 copies of Cy®3B, and a third luminescent label comprising 3 copies of ATRho6G. FIG. 10B shows a representative trace from a polypeptide sequencing reaction using a first luminescent label comprising 4 copies of Cy®3, a second luminescent label comprising 3 copies of Cy®3B, and a third luminescent label comprising 3 copies of C530NS. FIG. 10C shows a representative trace from a polypeptide sequencing reaction using a first luminescent label comprising 4 copies of Cy®3, a second luminescent label comprising 4 copies of Cy®3B, a third luminescent label comprising 2 copies of ATRho6G, and a fourth luminescent label comprising the luminescently labeled oligonucleotide structures from Example 4 comprising 8 copies of C530NS. FIG. 10C demonstrates that clear separation between the first, second, third, and fourth luminescent labels was achieved.

### Example 6

A polypeptide sequencing run was performed using amino acid recognition molecules labeled with a first luminescent label comprising 4 copies of Cy®3 (referred to as “TetraCy3”), a second luminescent label comprising 4 copies of Cy®3B (referred to as “TetraCy3B”), and a third luminescent label comprising 8 copies of Cy®3 (referred to as “OctaCy3”).

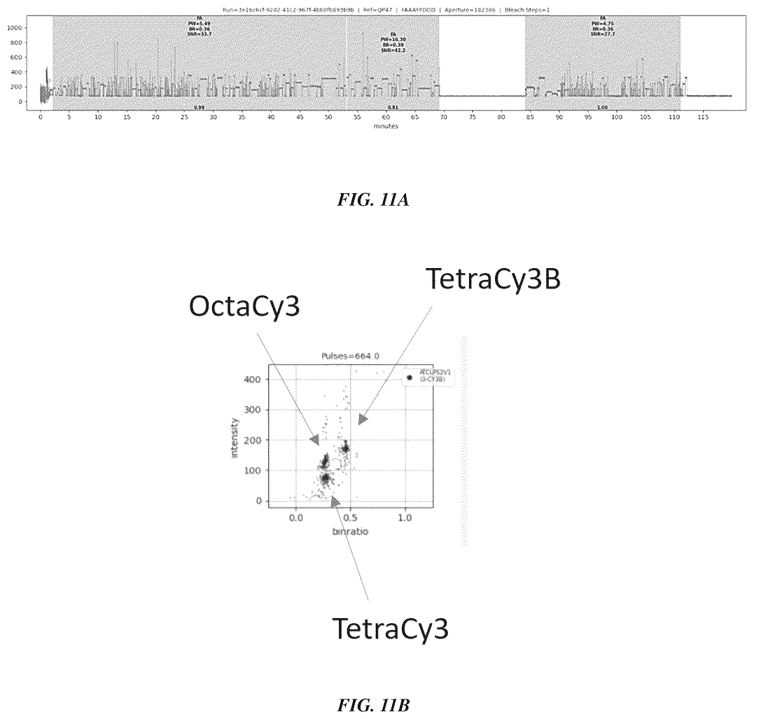

The polypeptide sequencing run was performed for a sample peptide having the sequence FAAAYPDDD (SEQ ID NO: 17). FIG. 11A shows a representative trace demonstrating that phenylalanine (F) was identified. FIG. 11B shows a plot of intensity vs. bin ratio. From FIG. 11B, it can be seen that each of the first, second, and third luminescent labels occupied distinct spatial regions of the plot.

### Example 7

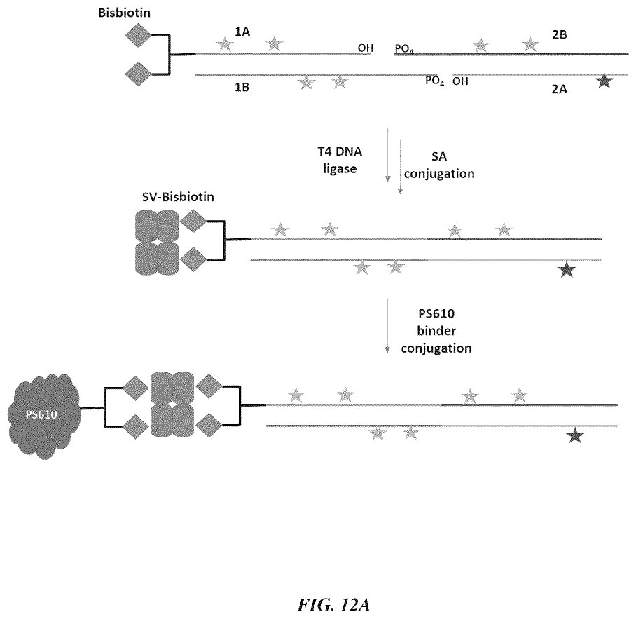

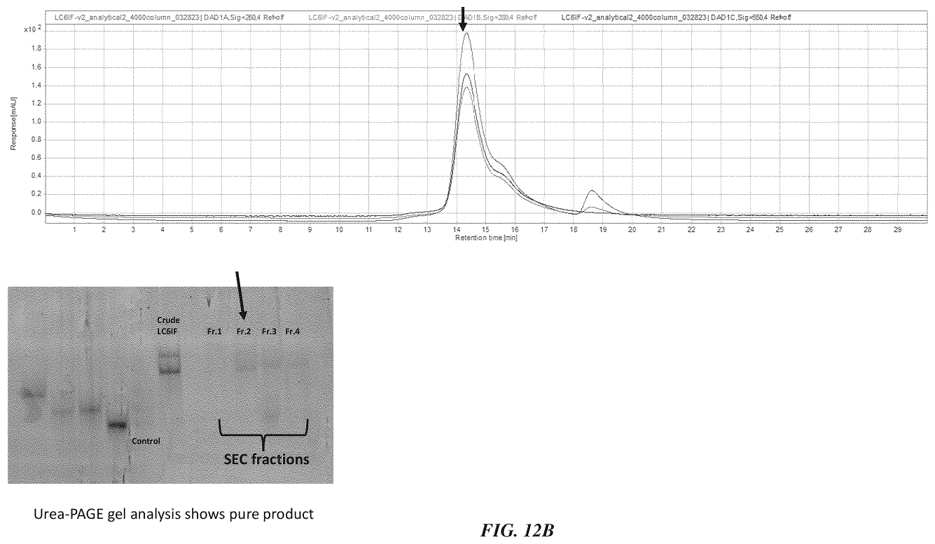

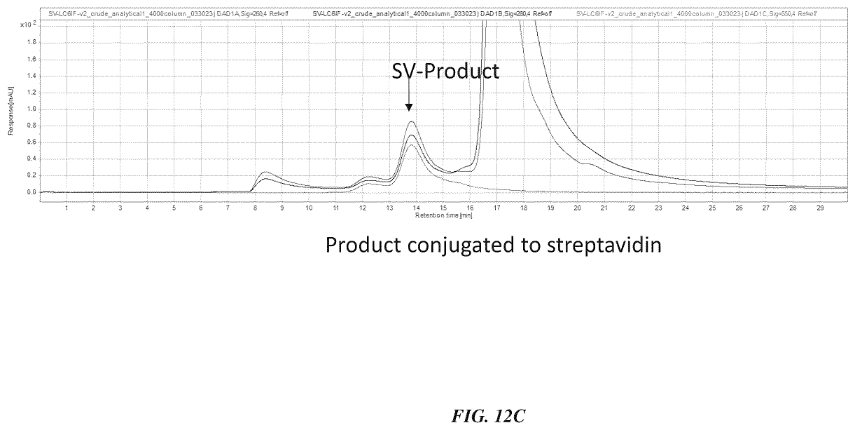

Luminescently labeled oligonucleotide structures comprising multiple luminescently labeled oligonucleotides were assembled by a stepwise ligation and conjugation approach, as schematically illustrated in FIG. 12A. As shown, two double-stranded oligonucleotides were prepared: a first formed by hybridized strands 1A and 1B, and a second formed by hybridized strands 2A and 2B. Each of strands 1A, 1B, and 2B contained two copies of internal Cy®3, and strand 2A contained one internal amine conjugated to iFluor® 570. Strand 1A contained a bis-biotin moiety at the 5′ end. Strands 1B and 2B contained a 5′-monophosphate in overhang regions of complementary sequence. Table 3 provides sequence information for the strands used in this example.

TABLE 3

| Oligo- | | |
|---|---|---|
| nucleotide | Name | Sequence* |
| 1A | K | /54/22/CCGAT/10/TACCCAT/ |
| | | 10/TACCGATATGAATCTTGCG |
| | | (SEQ ID NO: 12) |
| 1B | L | /MP/CGCTCGCAT/10/TAGATT |
| | | CAT/10/TTATCGGTTGGGTTC |
| | | GG (SEQ ID NO: 13) |
| 2A | M | CGATA/Z/TTACATCCGACTACA |
| | | GTTACCT (SEQ ID NO: 14) |
| 2B | N | /MP/AGCGAGGTAT/10/TACT |
| | | GTT/10/TAGTCGGATGTAATTA |
| | | TCG (SEQ ID NOs: 15 and |
| | | 16 from left to right) |

*Sequence notation: /54/: biotin (1-Dimethoxytrityloxy-2-(N-biotiny1-4-aminobutyl)-propyl-3-O-(2-cyanoethyl)-(N,N-diisopropyl)-phosphoramidite); /22/: symmetric doubler (1,3-bis-[5-(4,4′-dimethoxytrityloxy)pentylamido]propyl-2-[(2-cyanoethyl)-(N,N-diisopropyl)]-phosphoramidite); /10/: Cy®3 phosphoramidite; /MP/: 5′-monophosphate; and /Z/: internal amine phosphoramidite

The two double-stranded oligonucleotides were hybridized via the complementary overhang regions in strands 1B and 2B, followed by ligation using T4 DNA ligase to produce a single double-stranded oligonucleotide containing all six dyes. The ligated construct was purified by size-exclusion chromatography (FIG. 12B) and conjugated with streptavidin via the bis-biotin moiety of strand 1A (FIG. 12C). The streptavidin-conjugated construct was then conjugated to an amino acid recognition molecule (PS610) having a bis-biotin moiety (FIG. 12D).

### Example 8

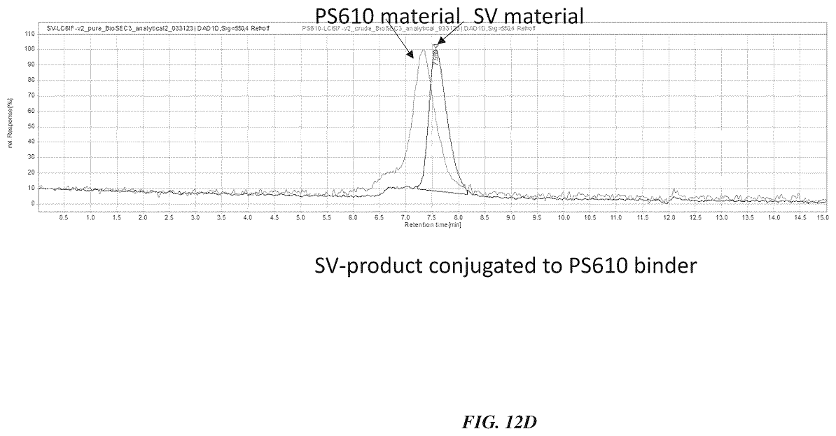

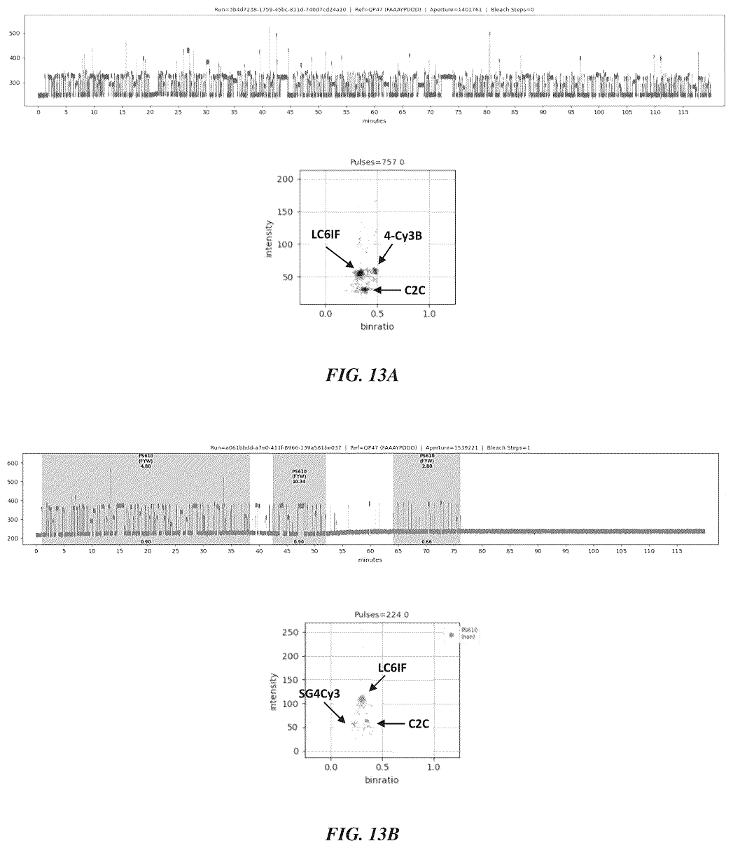

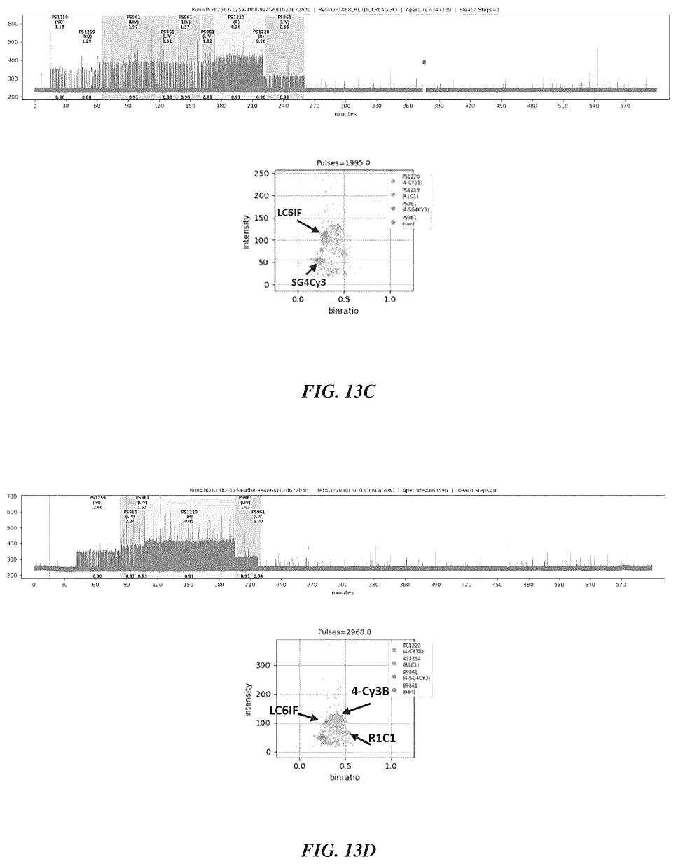

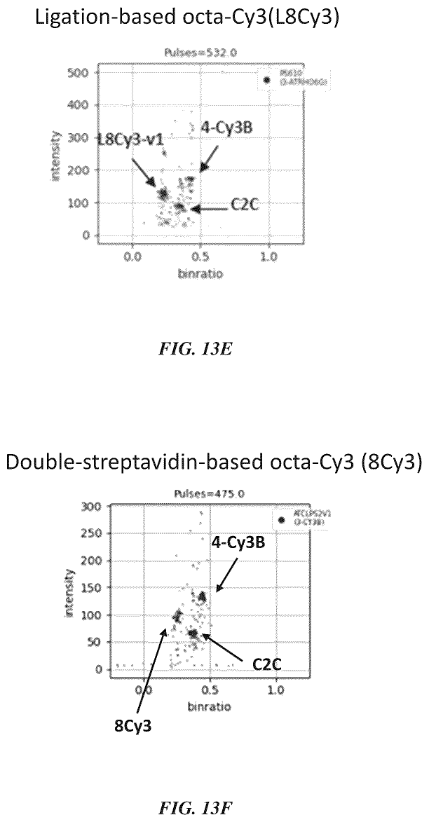

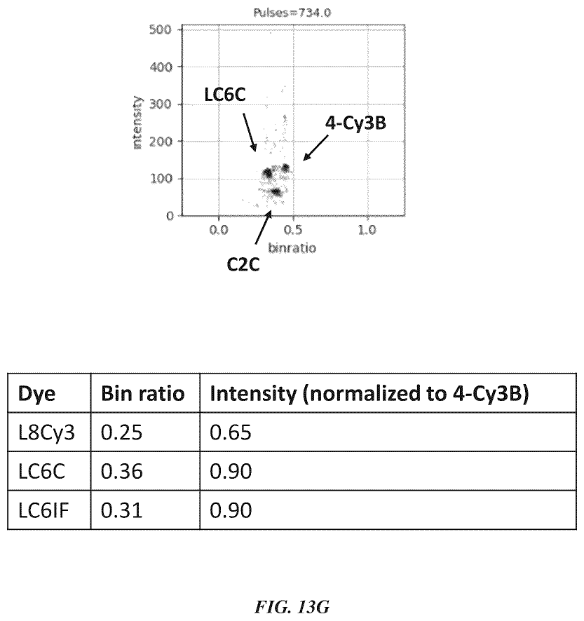

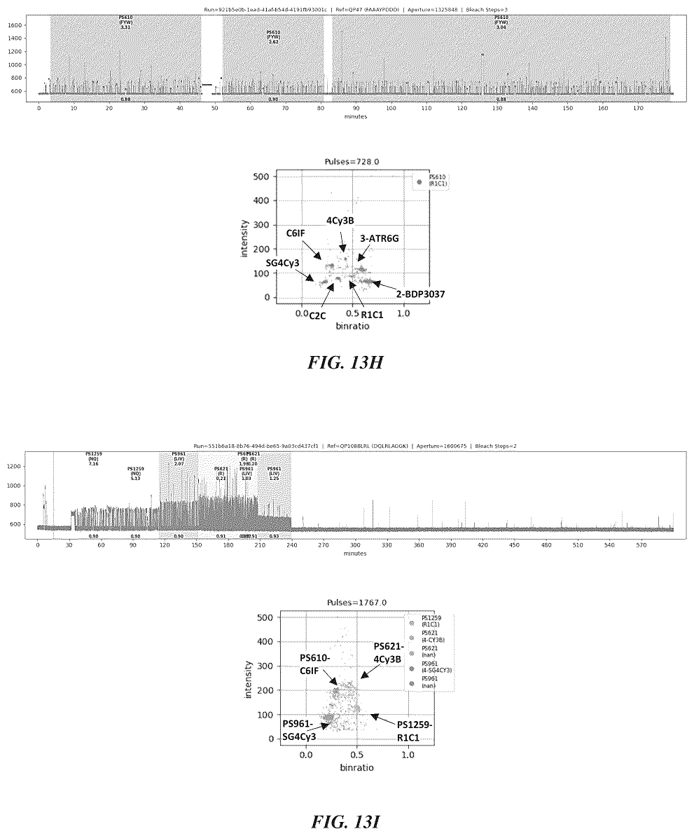

An amino acid recognition run was performed for a sample peptide (FAAAYPDDD (SEQ ID NO: 17)) using an amino acid recognition molecule labeled according to Example 7 (“LC6IF”). FIG. 13A shows a representative trace (top) demonstrating that phenylalanine (F) was identified, and a plot of intensity vs. bin ratio (bottom) showing distinct spatial separation of LC6IF, C2C (Example 2), and a recognition molecule having 4 copies of Cy®3B (“4-Cy3B”). FIG. 13B shows another representative trace (top) demonstrating that phenylalanine (F) was identified, and a plot of intensity vs. bin ratio (bottom) showing distinct spatial separation of LC6IF, C2C, and SG4Cy3 (Example 3).

Dynamic polypeptide sequencing reactions were performed for a sample peptide (DQLRLAGGK (SEQ ID NO: 20)) using a set of amino acid recognition molecules having distinct labels, including LC6IF. FIG. 13C shows a representative trace (top) demonstrating amino acid recognition during sample peptide degradation, and a plot of intensity vs. bin ratio (bottom) showing distinct spatial separation of LC6IF and SG4Cy3. FIG. 13D shows another representative trace (top) demonstrating amino acid recognition during sample peptide degradation, and a plot of intensity vs. bin ratio (bottom) showing distinct spatial separation of LC6IF, R1C1 (Example 1), and 4-Cy3B.

FIGS. 13E and 13F show _plots of intensity vs. bin ratio for dye sets that included C2C, 4-Cy3B, and a label having 8 copies of Cy®3 in a construct prepared by: ligation according to Example 7 (“L8Cy3”) (FIG. 13E); or double-streptavidin linkage according to Example 4 (“8Cy3”) (FIG. 13F). FIG. 13G shows a plot of intensity vs. bin ratio (top) and a table of corresponding values (bottom) for L8Cy3, LC6C, and LC6IF.

An amino acid recognition run was performed for a sample peptide (FAAAYPDDD (SEQ ID NO: 17)) using a set of seven amino acid recognition molecule having distinct labels, including LC6IF. FIG. 13H shows a representative trace (top) demonstrating that phenylalanine (F) was identified, and a plot of intensity vs. bin ratio (bottom) showing distinct spatial separation of seven distinctly labeled recognition molecules.

Dynamic polypeptide sequencing reactions were performed for a sample peptide (DQLRLAGGK (SEQ ID NO: 20)) using a set of amino acid recognition molecules having distinct labels, including LC6IF. FIG. 13I shows a representative trace (top) demonstrating amino acid recognition during sample peptide degradation, and a plot of intensity vs. bin ratio (bottom) showing distinct spatial separation of seven distinctly labeled recognition molecules.

The results in this example demonstrated that labeled oligonucleotides assembled by ligation (e.g., FIG. 3B) are as effective as labeled oligonucleotides assembled through binding molecule(s) (e.g., FIG. 3A) for resolving different clusters in a two-dimensional plot of intensity vs. bin ratio, and thus both constructs provide highly effective, differentiable luminescent labels.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A luminescently labeled oligonucleotide structure, the structure comprising:
a first single-stranded oligonucleotide comprising one or more first luminescent labels; and
a first complementary single-stranded oligonucleotide hybridized to the first single-stranded oligonucleotide, wherein the first complementary single-stranded oligonucleotide comprises one or more second luminescent labels,
wherein a closest distance between any first luminescent label and any second luminescent label is at least 10 nm.

2. The structure of claim 1, wherein the first luminescent label is different from the second luminescent label.

3. The structure of claim 1, wherein the first luminescent label is the same as the second luminescent label.

4. The structure of any one of claims 1-3, wherein the first luminescent label and the second luminescent label are fluorescent labels.

5. The structure of any one of claims 1-4, wherein the first luminescent label and the second luminescent label comprise a cyanine, a rhodamine, an ATTO-rhodamine, and/or a BODIPY dye.

6. The structure of any one of claims 1-5, wherein the first luminescent label comprises Cy®3, Cy®3B, ATRho6G, and/or C530NS.

7. The structure of any one of claims 1-6, wherein the first luminescent label comprises Cy®3.

8. The structure of any one of claims 1-6, wherein the first luminescent label comprises Cy®3B.

9. The structure of any one of claims 1-8, wherein the second luminescent label comprises Cy®3, Cy®3B, ATRho6G, and/or C530NS.

10. The structure of any one of claims 1-9, wherein the second luminescent label comprises Cy®3B.

11. The structure of any one of claims 1-9, wherein the second luminescent label comprises ATRho6G.

12. The structure of any one of claims 1-7 and 9-10, wherein the first luminescent label comprises Cy®3 and the second luminescent label comprises Cy®3B.

13. The structure of any one of claims 1-6, 8-9, and 11, wherein the first luminescent label comprises Cy®3B and the second luminescent label comprises ATRho6G.

14. The structure of any one of claims 1-13, wherein the first single-stranded oligonucleotide comprises two or more first luminescent labels, three or more first luminescent labels, or four or more first luminescent labels.

15. The structure of any one of claims 1-14, wherein the first complementary single-stranded oligonucleotide comprises two or more second luminescent labels, three or more second luminescent labels, or four or more second luminescent labels.

16. The structure of any one of claims 1-7 and 9-15, wherein the first single-stranded oligonucleotide comprises two first luminescent labels, wherein each first luminescent label comprises Cy®3.

17. The structure of any one of claims 1-10, 12, and 14-16, wherein the first complementary single-stranded oligonucleotide comprises one second luminescent label, wherein the second luminescent label comprises Cy®3B.

18. The structure of any one of claims 1-17, wherein the first single-stranded oligonucleotide further comprises one or more third luminescent labels, wherein the third luminescent label is different from the first luminescent label.

19. The structure of any one of claims 1-18, wherein the first complementary single-stranded oligonucleotide further comprises one or more fourth luminescent labels, wherein the fourth luminescent label is different from the second luminescent label.

20. The structure of any one of claims 1-19, wherein the structure has a length of at least 50, at least 70, or at least 100 base pairs.

21. The structure of any one of claims 1-20, wherein the first single-stranded oligonucleotide comprises a sequence that is at least 80% identical to Sequence A.

22. The structure of any one of claims 1-21, wherein the first complementary single-stranded oligonucleotide comprises a sequence that is at least 80% identical to Sequence B.

23. The structure of any one of claims 1-22, wherein the first single-stranded oligonucleotide comprises a sequence that is at least 80% identical to Sequence C.

24. The structure of any one of claims 1-23, wherein the first complementary single-stranded oligonucleotide comprises a sequence that is at least 80% identical to Sequence D.

25. The structure of any one of claims 1-24, wherein the closest distance between any first luminescent label and any second luminescent label is at least 11 nm, 12 nm, 13 nm, 14 nm, 15 nm, 16 nm, 17 nm, 18 nm, 19 nm, or 20 nm.

26. The structure of any one of claims 1-25, wherein the first single-stranded oligonucleotide is bound to a first binding molecule.

27. The structure of claim 26, wherein the first binding molecule comprises an avidin protein.

28. The structure of claim 27, wherein the avidin protein comprises streptavidin.

29. The structure of any one of claims 1-28, wherein the first single-stranded oligonucleotide comprises a biotin moiety.

30. The structure of claim 29, wherein the biotin moiety is a bis-biotin moiety.

31. The structure of any one of claims 26-30, further comprising an amino acid recognition molecule bound to the first binding molecule.

32. A luminescently labeled oligonucleotide structure, the structure comprising:
a first single-stranded oligonucleotide comprising two or more first luminescent labels; and
a first complementary single-stranded oligonucleotide hybridized to the first single-stranded oligonucleotide, wherein the first complementary single-stranded oligonucleotide comprises two or more first luminescent labels,
wherein the first luminescent label comprises a cyanine dye.

33. The structure of claim 32, wherein the cyanine dye comprises Cy®3.

34. The structure of any one of claims 32-33, wherein the first single-stranded oligonucleotide comprises a sequence that is at least 80% identical to Sequence E.

35. The structure of any one of claims 32-34, wherein the first complementary single-stranded oligonucleotide comprises a sequence that is at least 80% identical to Sequence F.

36. A luminescently labeled oligonucleotide structure, the structure comprising:
a first single-stranded oligonucleotide bound to a first binding molecule;
a first complementary single-stranded oligonucleotide hybridized to the first single-stranded oligonucleotide;
a second single-stranded oligonucleotide bound to the first binding molecule; and
a second complementary single-stranded oligonucleotide hybridized to the second single-stranded oligonucleotide,
wherein the first single-stranded oligonucleotide and/or the first complementary single-stranded oligonucleotide are conjugated to one or more first luminescent labels, and
wherein the second single-stranded oligonucleotide and/or the second complementary single-stranded oligonucleotide are conjugated to one or more second luminescent labels.

37. The structure of claim 36, wherein the first binding molecule comprises an avidin protein.

38. The structure of claim 37, wherein the avidin protein comprises streptavidin.

39. The structure of any one of claims 36-38, wherein the first single-stranded oligonucleotide and/or the second single-stranded oligonucleotide comprise a biotin moiety.

40. The structure of claim 39, wherein the biotin moiety comprises a bis-biotin moiety.

41. The structure of any one of claims 36-40, wherein the first single-stranded oligonucleotide and/or the first complementary single-stranded oligonucleotide are conjugated to two or more first luminescent labels.

42. The structure of any one of claims 36-41, wherein the first single-stranded oligonucleotide and the first complementary single-stranded oligonucleotide each are conjugated to two more first luminescent labels.

43. The structure of any one of claims 36-42, wherein the second single-stranded oligonucleotide and/or the second complementary single-stranded oligonucleotide are conjugated to two or more second luminescent labels.

44. The structure of any one of claims 36-43, wherein the second single-stranded oligonucleotide and the second complementary single-stranded oligonucleotide each are conjugated to two more second luminescent labels.

45. The structure of any one of claims 36-44, wherein the first luminescent label comprises Cy®3, Cy®3B, ATRho6G, and/or C530NS.

46. The structure of any one of claims 36-45, wherein the second luminescent label comprises Cy®3, Cy®3B, ATRho6G, and/or C530NS.

47. The structure of any one of claims 36-46, wherein the structure comprises at least four luminescent labels.

48. The structure of any one of claims 36-47, wherein the structure comprises at least eight luminescent labels.

49. The structure of any one of claims 36-48, wherein the second complementary single-stranded oligonucleotide is bound to a second binding molecule.

50. The structure of claim 49, further comprising a third single-stranded oligonucleotide bound to the second binding molecule.

51. The structure of claim 50, further comprising a third complementary single-stranded oligonucleotide hybridized to the third single-stranded oligonucleotide.

52. The structure of any one of claims 49-51, wherein the second binding molecule comprises an avidin protein.

53. The structure of claim 52, wherein the avidin protein comprises streptavidin.

54. The structure of any one of claims 50-53, further comprising an amino acid recognition molecule bound to the second binding molecule.

55. The structure of any one of claims 50-54, wherein the third single-stranded oligonucleotide comprises a biotin moiety.

56. The structure of claim 55, wherein the biotin moiety is a bis-biotin moiety.

57. The structure of any one of claims 50-56, wherein the third single-stranded oligonucleotide and/or the third complementary single-stranded oligonucleotide are conjugated to one or more third luminescent labels.

58. The structure of claim 57, wherein the third single-stranded oligonucleotide and/or the third complementary single-stranded oligonucleotide are conjugated to two or more third luminescent labels.

59. The structure of claim 58, wherein the third single-stranded oligonucleotide and the third complementary single-stranded oligonucleotide each are conjugated to two more third luminescent labels.

60. The structure of any one of claims 36-59, wherein the structure is at least 70 base pairs in length.

61. The structure of any one of claims 36-60, wherein the structure is at least 100 base pairs in length.

62. The structure of any one of claims 36-61, wherein the first single-stranded oligonucleotide comprises one or more isoguanine and/or isocytosine nucleotides and the second single-stranded oligonucleotide does not comprise one or more isoguanine and/or isocytosine nucleotides.

63. The structure of any one of claims 36-62, wherein the second single-stranded oligonucleotide comprises one or more isoguanine and/or isocytosine nucleotides and the first single-stranded oligonucleotide does not comprise one or more isoguanine and/or isocytosine nucleotides.

64. The structure of any one of claims 36-63, wherein the first single-stranded oligonucleotide or the second single-stranded oligonucleotide comprises at least one diaminopurine nucleotide.

65. A system, comprising:
an integrated device comprising a plurality of sample wells, wherein one or more sample wells are adapted to have a polypeptide immobilized to a surface thereof; and
one or more first amino acid recognition molecules bound to a first luminescent label comprising a first luminescently labeled oligonucleotide structure, the structure comprising:
a first single-stranded oligonucleotide comprising one or more first fluorophores; and
a first complementary single-stranded oligonucleotide hybridized to the first single-stranded oligonucleotide, wherein the first complementary single-stranded oligonucleotide comprises one or more second fluorophores,
wherein a closest distance between any first fluorophore and any second fluorophore is at least 10 nm.

66. The system of claim 65, further comprising one or more second amino acid recognition molecules bound to a second luminescent label.

67. The system of claim 66, wherein the first luminescent label has a first value for a first characteristic and the second luminescent label has a second value for the first characteristic, and wherein a percentage difference between the first value and the second value is at least 20%.

68. The system of claim 67, wherein the first characteristic comprises luminescent intensity and/or luminescent lifetime.

69. The system of any one of claims 65-68, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

70. The system of claim 69, wherein the first characteristic is different from the second characteristic.

71. The system of any one of claims 69-70, wherein the first characteristic comprises luminescent intensity and the second characteristic comprises luminescent lifetime.

72. The system of any one of claims 69-71, wherein the first ordered pair and the second ordered pair occupy different locations on a plot of the first characteristic versus the second characteristic.

73. The system of any one of claims 65-72, further comprising one or more third amino acid recognition molecules bound to a third luminescent label.

74. The system of claim 73, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

75. The system of claim 74, wherein the first characteristic comprises luminescent intensity and/or luminescent lifetime.

76. The system of any one of claims 73-75, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

77. The system of claim 76, wherein the first characteristic is different from the second characteristic.

78. The system of any one of claims 76-77, wherein the first characteristic comprises luminescent intensity and the second characteristic comprises luminescent lifetime.

79. The system of any one of claims 76-78, wherein the first ordered pair, the second ordered pair, and the third ordered pair occupy different locations on a plot of the first characteristic versus the second characteristic.

80. A system, comprising:
an integrated device comprising a plurality of sample wells, wherein one or more sample wells are adapted to have a polypeptide immobilized to a surface thereof; and
one or more first amino acid recognition molecules bound to first luminescent label comprising a luminescently labeled oligonucleotide structure, the structure comprising:
a first single-stranded oligonucleotide comprising two or more first fluorophores; and
a first complementary single-stranded oligonucleotide hybridized to the first single-stranded oligonucleotide, wherein the first complementary single-stranded oligonucleotide comprises two or more first fluorophores,
wherein the first fluorophore comprises a cyanine dye.

81. The system of claim 80, wherein the cyanine dye comprises Cy®3.

82. The system of any one of claims 80-81, wherein the first single-stranded oligonucleotide comprises a sequence that is at least 80% identical to Sequence E.

83. The system of nay one of claims 80-82, wherein the first complementary single-stranded oligonucleotide comprises a sequence that is at least 80% identical to Sequence F.

84. The system of any one of claims 80-83, further comprising one or more second amino acid recognition molecules bound to a second luminescent label.

85. The system of claim 84, wherein the first luminescent label has a first value for a first characteristic and the second luminescent label has a second value for the first characteristic, and wherein a percentage difference between the first value and the second value is at least 20%.

86. The system of claim 85, wherein the first characteristic comprises luminescent intensity and/or luminescent lifetime.

87. The system of any one of claims 84-86, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

88. The system of claim 87, wherein the first characteristic is different from the second characteristic.

89. The system of any one of claims 87-88, wherein the first characteristic comprises luminescent intensity and the second characteristic comprises luminescent lifetime.

90. The system of any one of claims 87-89, wherein the first ordered pair and the second ordered pair occupy different locations on a plot of the first characteristic versus the second characteristic.

91. The system of any one of claims 80-90, further comprising one or more third amino acid recognition molecules bound to a third luminescent label.

92. The system of claim 91, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

93. The system of claim 92, wherein the first characteristic comprises luminescent intensity and/or luminescent lifetime.

94. The system of any one of claims 91-93, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

95. The system of claim 94, wherein the first characteristic is different from the second characteristic.

96. The system of any one of claims 94-95, wherein the first characteristic comprises luminescent intensity and the second characteristic comprises luminescent lifetime.

97. The system of any one of claims 94-96, wherein the first ordered pair, the second ordered pair, and the third ordered pair occupy different locations on a plot of the first characteristic versus the second characteristic.

98. A system, comprising:
an integrated device comprising a plurality of sample wells, wherein one or more sample wells are adapted to have a polypeptide immobilized to a surface thereof; and
one or more first amino acid recognition molecules bound to a first luminescent label comprising a luminescently labeled oligonucleotide structure, the structure comprising:
a first single-stranded oligonucleotide bound to a first binding molecule;
a first complementary single-stranded oligonucleotide hybridized to the first single-stranded oligonucleotide;
a second single-stranded oligonucleotide bound to the first binding molecule; and
a second complementary single-stranded oligonucleotide hybridized to the second single-stranded oligonucleotide,
wherein the first single-stranded oligonucleotide and/or the first complementary single-stranded oligonucleotide are conjugated to one or more first fluorophores, and
wherein the second single-stranded oligonucleotide and/or the second complementary single-stranded oligonucleotide are conjugated to one or more second fluorophores.

99. The system of claim 98, further comprising one or more second amino acid recognition molecules bound to a second luminescent label.

100. The system of claim 99, wherein the first luminescent label has a first value for a first characteristic and the second luminescent label has a second value for the first characteristic, and wherein a percentage difference between the first value and the second value is at least 20%.

101. The system of claim 100, wherein the first characteristic comprises luminescent intensity and/or luminescent lifetime.

102. The system of any one of claims 99-101, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

103. The system of claim 102, wherein the first characteristic is different from the second characteristic.

104. The system of any one of claims 102-103, wherein the first characteristic comprises luminescent intensity and the second characteristic comprises luminescent lifetime.

105. The system of any one of claims 102-104, wherein the first ordered pair and the second ordered pair occupy different locations on a plot of the first characteristic versus the second characteristic.

106. The system of any one of claims 98-105, further comprising one or more third amino acid recognition molecules bound to a third luminescent label.

107. The system of claim 106, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

108. The system of claim 107, wherein the first characteristic comprises luminescent intensity and/or luminescent lifetime.

109. The system of any one of claims 106-108, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

110. The system of claim 109, wherein the first characteristic is different from the second characteristic.

111. The system of any one of claims 109-110, wherein the first characteristic comprises luminescent intensity and the second characteristic comprises luminescent lifetime.

112. The system of any one of claims 109-111, wherein the first ordered pair, the second ordered pair, and the third ordered pair occupy different locations on a plot of the first characteristic versus the second characteristic.

113. A method for determining chemical characteristics of a polypeptide, comprising:
contacting a polypeptide with one or more first amino acid recognition molecules bound to a first luminescent label comprising a luminescently labeled oligonucleotide structure, the structure comprising:
a first single-stranded oligonucleotide comprising one or more first fluorophores; and
a first complementary single-stranded oligonucleotide hybridized to the first single-stranded oligonucleotide, wherein the first complementary single-stranded oligonucleotide comprises one or more second fluorophores,
wherein a closest distance between any first fluorophore and any second fluorophore is at least 10 nm;
detecting a first series of signal pulses indicative of a first series of binding events between the one or more first amino acid recognition molecules and the polypeptide; and
determining at least one chemical characteristic for an amino acid of the polypeptide based on at least one characteristic of the first series of signal pulses.

114. The method of claim 113, further comprising contacting the polypeptide with one or more second amino acid recognition molecules bound to a second luminescent label.

115. The method of claim 114, wherein the first luminescent label has a first value for a first characteristic and the second luminescent label has a second value for the first characteristic, and wherein a percentage difference between the first value and the second value is at least 20%.

116. The method of claim 115, wherein the first characteristic comprises luminescent intensity and/or luminescent lifetime.

117. The method of any one of claims 114-116, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

118. The method of claim 117, wherein the first characteristic is different from the second characteristic.

119. The method of any one of claims 117-118, wherein the first characteristic comprises luminescent intensity and the second characteristic comprises luminescent lifetime.

120. The method of any one of claims 117-119, wherein the first ordered pair and the second ordered pair occupy different locations on a plot of the first characteristic versus the second characteristic.

121. The method of any one of claims 113-120, further comprising contacting the polypeptide with one or more third amino acid recognition molecules bound to a third luminescent label.

122. The method of claim 121, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

123. The method of claim 122, wherein the first characteristic comprises luminescent intensity and/or luminescent lifetime.

124. The method of any one of claims 121-123, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

125. The method of claim 124, wherein the first characteristic is different from the second characteristic.

126. The method of any one of claims 124-125, wherein the first characteristic comprises luminescent intensity and the second characteristic comprises luminescent lifetime.

127. The method of any one of claims 124-126, wherein the first ordered pair, the second ordered pair, and the third ordered pair occupy different locations on a plot of the first characteristic versus the second characteristic.

128. A method for determining chemical characteristics of a polypeptide, comprising:
contacting a polypeptide with one or first more amino acid recognition molecules bound to a first luminescent label comprising a luminescently labeled oligonucleotide structure, the structure comprising:
a first single-stranded oligonucleotide comprising two or more first fluorophores; and
a first complementary single-stranded oligonucleotide hybridized to the first single-stranded oligonucleotide, wherein the first complementary single-stranded oligonucleotide comprises two or more first fluorophores,
wherein the first luminescent label comprises a cyanine dye;
detecting a first series of signal pulses indicative of a first series of binding events between the one or more amino acid recognition molecules and the polypeptide; and
determining at least one chemical characteristic for an amino acid of the polypeptide based on at least one characteristic of the first series of signal pulses.

129. The method of claim 128, further comprising contacting the polypeptide with one or more second amino acid recognition molecules bound to a second luminescent label.

130. The method of claim 129, wherein the first luminescent label has a first value for a first characteristic and the second luminescent label has a second value for the first characteristic, and wherein a percentage difference between the first value and the second value is at least 20%.

131. The method of claim 130, wherein the first characteristic comprises luminescent intensity and/or luminescent lifetime.

132. The method of any one of claims 129-131, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

133. The method of claim 132, wherein the first characteristic is different from the second characteristic.

134. The method of any one of claims 132-133, wherein the first characteristic comprises luminescent intensity and the second characteristic comprises luminescent lifetime.

135. The method of any one of claims 132-134, wherein the first ordered pair and the second ordered pair occupy different locations on a plot of the first characteristic versus the second characteristic.

136. The method of any one of claims 132-135, further comprising contacting the polypeptide with one or more third amino acid recognition molecules bound to a third luminescent label.

137. The method of claim 136, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

138. The method of claim 137, wherein the first characteristic comprises luminescent intensity and/or luminescent lifetime.

139. The method of any one of claims 136-138, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

140. The method of claim 139, wherein the first characteristic is different from the second characteristic.

141. The method of any one of claims 139-140, wherein the first characteristic comprises luminescent intensity and the second characteristic comprises luminescent lifetime.

142. The method of any one of claims 139-141, wherein the first ordered pair, the second ordered pair, and the third ordered pair occupy different locations on a plot of the first characteristic versus the second characteristic.

143. A method for determining chemical characteristics of a polypeptide, comprising:
contacting a polypeptide with one or more first amino acid recognition molecules bound to a first luminescent label comprising a luminescently labeled oligonucleotide structure, the structure comprising:
a first single-stranded oligonucleotide bound to a first binding molecule;
a first complementary single-stranded oligonucleotide hybridized to the first single-stranded oligonucleotide;
a second single-stranded oligonucleotide bound to the first binding molecule; and
a second complementary single-stranded oligonucleotide hybridized to the second single-stranded oligonucleotide,
wherein the first single-stranded oligonucleotide and/or the first complementary single-stranded oligonucleotide are conjugated to one or more first fluorophores, and
wherein the second single-stranded oligonucleotide and/or the second complementary single-stranded oligonucleotide are conjugated to one or more second fluorophores
detecting a first series of signal pulses indicative of a first series of binding events between the one or more amino acid recognition molecules and the polypeptide; and
determining at least one chemical characteristic for an amino acid of the polypeptide based on at least one characteristic of the first series of signal pulses.

144. The method of claim 143, further comprising contacting the polypeptide with one or more second amino acid recognition molecules bound to a second luminescent label.

145. The method of claim 144, wherein the first luminescent label has a first value for a first characteristic and the second luminescent label has a second value for the first characteristic, and wherein a percentage difference between the first value and the second value is at least 20%.

146. The method of claim 145, wherein the first characteristic comprises luminescent intensity and/or luminescent lifetime.

147. The method of any one of claims 144-146, wherein the first luminescence label has a first ordered pair of characteristics comprising a first value of a first characteristic and a first value of a second characteristic, wherein the second luminescence label has a second ordered pair of characteristics comprising a second value of the first characteristic and a second value of the second characteristic, and wherein the first ordered pair and the second ordered pair differ in at least one of the respective values of the first and/or second characteristics.

148. The method of claim 147, wherein the first characteristic is different from the second characteristic.

149. The method of any one of claims 147-148, wherein the first characteristic comprises luminescent intensity and the second characteristic comprises luminescent lifetime.

150. The method of any one of claims 147-149, wherein the first ordered pair and the second ordered pair occupy different locations on a plot of the first characteristic versus the second characteristic.

151. The method of any one of claims 143-150, further comprising contacting the polypeptide with one or more third amino acid recognition molecules bound to a third luminescent label.

152. The method of claim 151, wherein the first luminescent label has a first value for a first characteristic, the second luminescent label has a second value for the first characteristic, and the third luminescent label has a third value for the first characteristic, and wherein a minimum percentage difference between the first value, the second value, and the third value is at least 20%.

153. The method of claim 152, wherein the first characteristic comprises luminescent intensity and/or luminescent lifetime.

154. The method of any one of claims 151-153, wherein the first luminescence label has a first ordered pair of characteristics comprising a first value of a first characteristic and a first value of a second characteristic, the second luminescence label has a second ordered pair of characteristics comprising a second value of the first characteristic and a second value of the second characteristic, and the third luminescence label has a third ordered pair of characteristics comprising a third value of the first characteristic and a third value of the second characteristic, and wherein the first ordered pair, the second ordered pair, and the third ordered pair differ in at least one of the respective values of the first and/or second characteristics.

155. The method of claim 154, wherein the first characteristic is different from the second characteristic.

156. The method of any one of claims 154-155, wherein the first characteristic comprises luminescent intensity and the second characteristic comprises luminescent lifetime.

157. The method of any one of claims 154-156, wherein the first ordered pair, the second ordered pair, and the third ordered pair occupy different locations on a plot of the first characteristic versus the second characteristic.

158. A system, comprising:
a first luminescent label having a first ordered pair of characteristics comprising a first value of a first characteristic and a first value of a second characteristic;
a second luminescent label having a second ordered pair of characteristics comprising a second value of the first characteristic and a second value of the second characteristic; and
a third luminescent label having a third ordered pair of characteristics comprising a third value of the first characteristic and a third value of the second characteristic;
wherein the first ordered pair, the second ordered pair, and the third ordered pair differ from one another in at least one of the respective values of the first and/or second characteristics.

159. The system of claim 158, wherein the first characteristic is different from the second characteristic.

160. The system of any one of claims 158-159, wherein the first characteristic relates to luminescent intensity.

161. The system of any one of claims 158-160, wherein the second characteristic relates to luminescent lifetime.

162. The system of any one of claims 158-161, wherein the first luminescent label, the second luminescent label, and the third luminescent label occupy different locations on a plot of the first characteristic versus the second characteristic.

163. The system of any one of claims 158-162, wherein at least one of the first luminescent label, the second luminescent label, and the third luminescent label is a luminescently labeled oligonucleotide structure comprising a first single-stranded oligonucleotide comprising one or more first luminescent labels and a first complementary single-stranded oligonucleotide comprising one or more second luminescent labels.

164. The system of any one of claims 158-163, wherein the first luminescent label comprises R1C1.

165. The system of any one of claims 158-164, wherein the second luminescent label comprises C2C.

166. The system of any one of claims 158-165, wherein the third luminescent label comprises SG4Cy3.

167. The system of any one of claims 158-166, further comprising a fourth luminescent label having a fourth value of the first luminescence characteristic and a fourth value of the second luminescence characteristic, wherein a minimum percentage difference between the first value, the second value, the third value, and the fourth value of the first luminescence characteristic is at least 20%, and wherein a minimum percentage difference between the first value, the second value, the third value, and the fourth value of the second luminescence characteristic is at least 20%.

168. The system of claim 167, wherein the fourth luminescent label comprises at least one copy of ATRho6G.

169. The system of any one of claims 158-168, further comprising a fifth luminescent label having a fifth value of the first luminescence characteristic and a fifth value of the second luminescence characteristic, wherein a minimum percentage difference between the first value, the second value, the third value, the fourth value, and the fifth value of the first characteristic is at least 20%, and wherein a minimum percentage difference between the first value, the second value, the third value, the fourth value, and the fifth value of the second characteristic is at least 20%.

170. The system of claim 169, wherein the fifth luminescent label comprises at least one copy of Cy®3B.

171. A system, comprising:
a first luminescent label having a first bin ratio value;
a second luminescent label having a second bin ratio value; and
a third luminescent label having a third bin ratio value;
wherein a minimum difference between the first bin ratio value, the second bin ratio value, and the third bin ratio value of the first luminescence characteristic is at least 0.1.

172. The system of claim 171, wherein the first luminescent label comprises R1C1.

173. The system of any one of claims 171-172, wherein the second luminescent label comprises C2C.

174. The system of any one of claims 171-173, wherein the third luminescent label comprises SG4Cy3, at least one copy of ATRho6G, and/or at least one copy of Cy®3B.

175. The system of any one of claims 171-174, wherein the minimum difference is at least 0.2.

176. A method, comprising:
providing a first luminescent label having a first ordered pair of characteristics comprising a first value of a first characteristic and a first value of a second characteristic;
providing a second luminescent label having a second ordered pair of characteristics comprising a second value of the first characteristic and a second value of the second characteristic;
providing a third luminescent label comprising a luminescently labeled oligonucleotide structure comprising a first single-stranded oligonucleotide comprising one or more first fluorophores and a first complementary single-stranded oligonucleotide comprising one or more second fluorophores, wherein the third luminescent label has a third ordered pair of characteristics comprising a third value of the first characteristic and a third value of the second characteristic; and
modifying the numbers and/or identities of the one or more first fluorophores and/or the one or more second fluorophores such that the first ordered pair, the second ordered pair, and the third ordered pair differ from one another in at least one of the respective values of the first and/or second characteristics.

177. The method of claim 176, wherein the first characteristic is different from the second characteristic.

178. The method of any one of claims 176-177, wherein the first characteristic comprises luminescent intensity and the second characteristic comprises luminescent lifetime.

179. The method of any one of claims 176-178, wherein the first ordered pair, the second ordered pair, and the third ordered pair occupy different locations on a plot of the first characteristic versus the second characteristic.

180. A method of preparing a luminescently labeled reaction component, the method comprising:
ligating a first double-stranded oligonucleotide comprising a first luminescent label to a second double-stranded oligonucleotide comprising a second luminescent label, wherein the first double-stranded oligonucleotide comprises a first binding moiety;
contacting the ligated double-stranded oligonucleotides with a multivalent protein that binds the first binding moiety to form a complex comprising the ligated double-stranded oligonucleotides and the multivalent protein; and
contacting the complex with a reaction component comprising a second binding moiety, wherein the multivalent protein of the complex binds the second binding moiety to form a luminescently labeled reaction component.

181. The method of claim 180, wherein the first double-stranded oligonucleotide comprises two or more first luminescent labels.

182. The method of claim 180 or 181, wherein the second double-stranded oligonucleotide comprises two or more second luminescent labels.

183. The method of any one of claims 180-182, wherein the first luminescent label is different from the second luminescent label.

184. The method of any one of claims 180-182, wherein the first luminescent label is the same as the second luminescent label.

185. The method of any one of claims 180-184, wherein the first and second luminescent labels comprise cyanine dyes.

186. The method of any one of claims 180-185, wherein the first and second luminescent labels are each independently selected from the group consisting of Cy®3, Cy®3B, ATRho6G, C530NS, and iFluor® 570.

187. The method of any one of claims 180-186, wherein the first or second double-stranded oligonucleotide comprises a third luminescent label that is different from the first and second luminescent labels.

188. The method of claim 187, wherein each of the first and second luminescent labels is Cy®3, and wherein the third luminescent label is selected from the group consisting of Cy®3B, ATRho6G, C530NS, and iFluor® 570.

189. The method of claim 188, wherein the third luminescent label is iFluor® 570.

190. The method of any one of claims 180-189, further comprising, prior to the ligating:
contacting the first double-stranded oligonucleotide with the second double-stranded oligonucleotide under hybridization conditions,
wherein the first double-stranded oligonucleotide comprises a first overhang,
wherein the second double-stranded oligonucleotide comprises a second overhang that is complementary to the first overhang, and
wherein the hybridization conditions are sufficient to hybridize the first overhang of the first double-stranded oligonucleotide to the second overhang of the second double-stranded oligonucleotide.

191. The method of any one of claims 180-190, wherein the first and second luminescent labels of the luminescently labeled reaction component are separated from one another by a distance of at least 10 nm.

192. The method of any one of claims 180-191, wherein the first double-stranded oligonucleotide comprises one or more isoguanine and/or isocytosine nucleotides, and the second double-stranded oligonucleotide does not comprise one or more isoguanine and/or isocytosine nucleotides.

193. The method of any one of claims 180-192, wherein the second double-stranded oligonucleotide comprises one or more isoguanine and/or isocytosine nucleotides, and the first double-stranded oligonucleotide does not comprise one or more isoguanine and/or isocytosine nucleotides.

194. The method of any one of claims 180-193, wherein the first or second double-stranded oligonucleotide comprises at least one diaminopurine nucleotide.

195. The method of any one of claims 180-194, wherein the first and second binding moieties are first and second biotin moieties, respectively.

196. The method of claim 195, wherein at least one of the first and second biotin moieties is a bis-biotin moiety.

197. The method of any one of claims 180-196, wherein the multivalent protein is an avidin protein.

198. The method of claim 197, wherein the avidin protein comprises streptavidin.

199. The method of any one of claims 180-198, wherein the reaction component comprises an amino acid recognition molecule.
